(19) United States
(12) Patent Application Publication
(10) Pub. No.: US 2008/0233132 A1
(43) Pub. Date: Sep. 25, 2008

(54) MULTIPLE SCLEROSIS THERAPY

(76) Inventors: Stephen D. Miller, Oak Park, IL (US); Terra J. Frederick, Chicago, IL (US)

Correspondence Address: WILSON SONSINI GOODRICH & ROSATI, 650 PAGE MILL ROAD, PALO ALTO, CA, 94304-1050, US

(21) Appl. No.: 11/934,641

(22) Filed: Nov. 2, 2007

Related U.S. Application Data
(60) Provisional application No. 60/864,295, filed on Nov. 3, 2006.

(51) Int. Cl. A61K 39/395; A61K 36/00; C12N 5/06; A61K 38/02; A61P 37/00; A61K 31/55; A61K 31/7088

(52) U.S. Cl. 424/172.1; 514/2; 424/93.1; 514/44; 424/130.1; 435/373; 514/212.04

(57) ABSTRACT

The present invention relates to methods for treating multiple sclerosis by combining immunotherapy with myelin repair.

### CROSS-REFERENCE

This application claims the benefit of U.S. Provisional Application No. 60/864,295, filed Nov. 3, 2006, which is incorporated herein by reference in its entirety.

### BACKGROUND OF THE INVENTION

Multiple sclerosis (MS) is a demyelinating disease of the central nervous system (CNS) with clinical deficits ranging from relapsing-remitting to chronic-progressive patterns of expression. Although the etiology of MS is unknown, autoreactive CD4⁺ T cell responses mediate inflammatory damage against myelin and oligodendrocytes. (Bruck et al., J. Neurol. Sci. 206:181-185 (2003)). CNS lesions have focal areas of myelin damage and are also associated with axonal pathology, neuronal distress, and astroglial scar formation. (Compston et al., Lancet. 359:1221-1231 (2002)). Clinical presentation includes various neurological dysfunctions including blindness, paralysis, loss of sensation, as well as coordination and cognitive deficits.

Damage or injury to myelin has severe consequences on conduction velocity and the vulnerability of neurons to axonal destruction. There is a correlation between axon loss and progressive clinical disability and intact myelin is important in the maintenance of axonal integrity (Dubois-Dalcq et al., Neuron. 48, 9-12 (2005)). Spontaneous remyelination occurs during the early phases of human MS (Prineas et al., Ann. Neurol. 33:137-151 (1993)), and has been shown to restore neurophysiological function in animal models of MS (Stangel et al., Prog. Neurobiol. 68:361-376 (2002)). However, persistent CNS inflammation and the failure of myelin repair during later stages of the disease ultimately lead to permanent debilitation (Bruck et al., J. Neurol. Sci. 206:181-185 (2003), Keirstead et al., Func. Roles of Glial Cells in Health and Dis. 468:183-197 (1999)).

Genetic evidence has linked MS susceptibility to the major histocompatibility complex (MHC) class II allele human leukocyte antigen (HLA)-DR2 haplotype, which strongly implicates a role for CD4⁺ T cells in MS pathogenesis (Oksenberg et al., JAMA 270:2363-2369 (1993); Olerup et al., Tissue Antigens 38:1-3 (1991)). It is generally believed that autoreactive T cell responses directed against myelin and oligodendrocytes produce inflammatory CNS lesions and neurological dysfunction during the early phases of MS, through processes including secretion of proinflammatory (e.g. Th1 and Th17) cytokines, which stimulates microglia and astrocytes, recruit other inflammatory cells, and induce antibody production by B cells (Prat et al., J. Rehabil. Res. Dev. 39:187-199 (2002); Hemmer et al., Nat. Rev. Neurosci. 3:291-301 (2002)).

Currently available treatments for relapsing MS, which include interferon-β, glatiramer acetate and mitoxanthrone, typically nonspecifically suppress the immunological response and marginally decrease the development of new lesions in some patients, providing little benefit in the progression of disease and do not typically induce myelin repair (Lubetzki et al., Curr. Opin. Neurol. 18:237-244 (2005). It is well accepted that adult oligodendrocyte progenitor cells are responsible for remyelination (Dawson et al., Mol. Cell. Neurosci. 24:476-488 (2003), Watanabe et al., J. Neurosci. Res. 69:826-836 (2002), Keirstead et al., J. Neuropathol. Exp. Neurol 56:1191-1201 (1997), Gensert et al., Neuron. 19:197-203 (1997)), and thus, the failure of remyelination is most likely associated with deficiencies in the generation of mature oligodendrocytes, their ability to myelinate, and/or neurodegeneration and axons that are not receptive to myelination (Bjartmar et al., Curr. Opin. Neurol. 14:271-278 (2002), Papadopoulos et al., Exp. Neurol. 197:373-385 (2006)). Thus, there exists a need for developing therapeutic strategies to suppress the autoimmune response and promote remyelination.

Suppression of the autoimmune response may be based upon the premise that engagement of the T cell receptor (TCR) in the absence of required costimulatory signals generally results in a TCR signal of insufficient strength to lead to T cell activation, but a signal of sufficient strength to result in the induction of long-term anergy, tolerance or cellular depletion. Though these immunotherapies typically result in suppression of the underlying autoimmune component of the disease process and the amelioration of continued myelin destruction, the animals are typically left with a clinical paralytic deficit from which they do not recover, presumably due to failure to repair damaged myelin. To repair the damage of repeated immunological attacks, sufficient numbers of oligodendrocytes must be replaced and these cells must efficiently contact and remyelinate denuded axons. Effective treatment for MS should address both facets of the disease using combinatorial treatments, which are geared to both suppress ongoing autoimmune inflammatory responses in an antigen-specific fashion and promote remyelination.

Many factors are known to enhance oligodendrocyte generation and myelination in vitro and in vivo. Thus, there are a number of potential oligodendrocyte regenerative strategies that may be used in combination with immunoregulatory strategies to enhance remyelination. Such strategies include γ-secretase inhibition or transfer of oligodendrocyte progenitor cells (OPCs).

Active γ-secretase is a multi-protein complex involved in proteolysis within the membrane. Active γ-secretase is typically composed of a complex of four proteins, of which presenilin (PS) is thought to provide the active site through two highly-conserved aspartates, D257 and D385, located within transmembrane domains of the protein. To become active, immature PS is processed and incorporated into a complex with other proteins to become stabilized. This usually includes a proteolytic cleavage by an enzyme termed “presenilinase” that produces N-terminal fragment and C-terminal fragments that remain associated with one another in the mature protease, with each fragment containing one of the two essential aspartates. The complex of four proteins can reconstitute the γ-secretase activity

PS alone itself is also often referred to as “γ-secretase” based on its proposed role as the active core of the complex.

γ-secretase has many known targets, such as integral membrane protein substrates. Notch is a substrate of γ-secretase. Notch, whose biological activity typically depends both on its function as a cell surface receptor and a transcriptional regulator, is typically cleaved at the S2 site by proteases of the ADAM family upon ligand binding. Cleavage usually results in release of the extracellular domain. The remaining truncated transmembrane form of Notch is then typically subject to cleavage at two sites within the membrane S3 and S4 sites, which are targets of γ-secretase. The cleaved Notch can translocate to the nucleus where it activates Notch target genes, which includes regulators of a host of cellular processes including the inhibition of neuronal differentiation, oligodendrocyte differentiation, and myelination.

Some of the identified targets of γ-secretase are ligands of receptors that are themselves known targets for γ-secretase, such as the Notch ligands Jagged and Delta. Other identified substrates of γ-secretase cleavage that are likely regulators of CNS myelination include N-cadherin, the cysteine-rich domain isoform of neuregulin-1 (CRD-NRG), and the neuregulin receptor erbB4. The neuregulins (NRGs) are a large family of signaling proteins that includes multiple soluble and transmembrane isoforms encoded by at least four genes. Expressed by a variety of neurons, they may have complex, context-dependent effects on the development of myelinating glia, such as promoting proliferation of precursors or maturation of oligodendrocytes (OLs). They may also provide an axon-derived survival signal for developing OLs, perhaps in conjunction with integrin ligands such as laminin-2. They may mediate these effects through transmembrane receptor tyrosine kinases of the erbB family, such as heterodimers of erbB2/erbB3 and erbB2/erbB4.

The present invention provides methods for treating neuropathies by combining immunoregulation strategies with myelin repair and/or axonal protection strategies, providing a synergistic therapeutic effect.

### SUMMARY OF THE INVENTION

The present invention comprises compositions and methods providing combinatorial delivery or administration of biologically active agents to effect immunoregulation as well as promote myelin repair, remyelination and/or axonal maintenance, protection or regeneration. In some embodiments, administration of an immunoregulatory agent is before, concurrent to or subsequent to administration of agent(s) that effect myelin repair, remyelination and/or axonal protection (collectively “axonal protection”). The present invention is directed to multimodal therapeutic methods in which the administration of an agent for immunomodulation is supplemented by administration of other therapeutic modalities for effecting myelin repair, remyelination and/or axonal protection.

Autoimmune suppression, myelin repair, and axon protection/re-growth represent key objectives in the design of a successful treatment regimen (FIG. 1). In some embodiments, the immunoregulatory component specifically target myelin-specific T cells (FIG. 2).

The combinatorial or co-administration of agents directed to different end points can enhance or produce a synergistic effect in subjects suffering from a neuropathy or neuropathy related condition. In other words one or more agents are delivered in combination to produce an enhanced or synergistic therapeutic result. Thus agents can be administered that modulate an immune response, along with agents that result in myelin repair, remyelination and/or axonal regeneration.

In some embodiments, an agent may have more than one effect (e.g., immunoregulation and enhancing myelin repair), in which case the degree of therapeutic synergism or effect can be enhanced as well. In other embodiments, one or more agents are administered which are directed to the same first endpoint (e.g., immunomodulation), and such agents are co-administered with one or more agents that are directed to the same second endpoint (e.g., myelin repair or axonal protection). The combinatorial regime of agents directed to different endpoints results in a synergistic therapeutic effect in subjects suffering from a neuropathy or related condition.

In one aspect of the invention, a composition for treating a demyelinating condition comprising: a) a therapeutically effective amount of a first agent, wherein the first agent is immunomodulatory; and, b) a therapeutically effective amount of a second agent, wherein the second agent promotes myelin repair, and administering the first and second agents result in a synergistic therapeutic effect for treating the demyelinating condition, is provided. In some embodiments, the first and second agents are present in synergistic amounts.

The present invention also provides a composition for treating a demyelinating condition comprising: a) a therapeutically effective amount of a first agent, wherein the first agent is immunomodulatory; b) a therapeutically effective amount of a second agent, wherein the second agent promotes oligodendrocyte differentiation, and, c) a therapeutically effective amount of a third agent, wherein the third agent promotes oligodendrocyte proliferation, and administering the first, second and third agents result in a synergistic therapeutic effect for treating said demyelinating condition.

The present invention also provides methods for treating a neuropathy comprising the compositions described herein. In some embodiments, the present invention provides a method for treating a demyelinating condition comprising administering to a subject in need thereof: a) a therapeutically effective amount of a first agent, wherein the first agent is immunomodulatory; and, b) a therapeutically effective amount of a second agent, wherein the second agent promotes remyelination, and administering the first and second agents result in a synergistic therapeutic effect in promoting remyelination. In some embodiments, the present invention provides a method of promoting remyelination comprising: a) contacting a cell in a co-culture with a first agent, wherein the first agent is immunomodulatory, and, b) contacting the cell with a second agent, wherein the second agent promotes remyelination, and contacting the cell with the first and second agents result in a synergistic effect in promoting remyelination. The methods of the present invention may occur in vitro or in vivo. Also provided herein is a method for treating a demyelinating condition comprising administering to a subject in need thereof: a) a therapeutically effective amount of a first agent, wherein said first agent is immunomodulatory; b) a therapeutically effective amount of a second agent, wherein said second agent promotes oligodendrocyte differentiation, and, c) a therapeutically effective amount of a third agent, wherein said third agent promotes oligodendrocyte proliferation, wherein administering the first, second and third agents result in a synergistic therapeutic effect for treating the demyelinating condition. In one aspect, methods for treating a neuropathy comprising delivering a therapeutically effective amount of a biologically active agent that modulates a T cell-mediated immune response in a subject and concurrently a therapeutically effective amount of a biologically active agent that promotes myelin repair, remyelination and/or axonal protection.

In some embodiments, the first agent and said second agent are not administered concurrently. In some embodiments, the first agent is administered concurrent with said second agent. In some embodiments, the composition is administered to treat multiple sclerosis. In another aspect of the present invention, the composition may provide a synergistic effect is more than 1 fold than the therapeutic effect of said first agent alone or said second agent alone. In another aspect, the first agent of the composition may suppress the autoimmune response. In some embodiments, the first agent targets T-cells, plasma cells, or macrophages. In some embodiments, the first agent inhibits T-cell receptor signaling in an autoimmune response.

The first or second agent of the present invention may be selected from the group consisting of: an altered peptide ligand, peptide-coupled cell, antisense molecule, siRNA, aptamer, small molecule and antibody. The first agent is specific for a ligand, or its receptor, wherein said ligand is selected from the group consisting of: CD80, CD86, CD28, CD40L, CD3, CD4, CD22, CD25, CD40, CD44, CD45, CD45RB, CD49, CD62, CD69, and CD154. In preferred embodiments, the first agent is a CD80 antibody or CD3 antibody. In some embodiments, the second agent inhibits Notch signaling. In other embodiments, the second agent is an IgM antibody or a γ-secretase inhibitor. The second agent may be selected from a group consisting of: DAPT, Ly411575, III-31-C, and rHIgM22.

In some embodiments, the biologically active compound, or agent, is specific for an antigen associated with T cell proliferation, differentiation or regulation. In some embodiments, the biologically active compound is an altered peptide ligand, peptide-coupled cell, antibody, peptide, aptamer, antisense molecule, siRNA, ribozyme, small molecule or chemical compound, or functional variants thereof. In some embodiments, the first agent administered is specific for a ligand, or its receptor, wherein said ligand is selected from the group consisting of: CD80, CD86, CD28, CD40L, CD3, CD4, CD22, CD25, CD40, CD44, CD45, CD45RB, CD49, CD62, CD69, and CD154. In some embodiments, the first agent is a CD80 antibody or a CD3 antibody. The second agent that may be administered, may inhibit Notch signaling. In some embodiments, the second agent is an IgM antibody or a γ-secretase inhibitor. The second agent may be selected from a group consisting of DAPT, Ly411575, III-31-C, and rHIgM22.

In one embodiment, a combinatorial treatment process is directed to treating a subject in need thereof, where such a process comprises delivering a therapeutically effective amount of a compound that is specific for a cluster of differentiation (CD) protein involved in T cell proliferation, differentiation or regulation thereof, wherein said method further comprises also delivering a therapeutically effective amount of a compound that promotes remyelination, myelination, and/or axonal protection. In one embodiment, the compound is a protein kinase C (PKC) pathway inhibitor. In a further embodiment, the compound inhibits a PKC theta pathway in immune cells. In yet other embodiments, the compound inhibits the B-lymphocyte activation antigen B7-1 (CD80).

In some aspects of the invention, one or more methods of the invention comprise combinatorial treatment which can be varied with respect to the sequence of delivery to comprise in any order, delivering one or more compounds to effect an (1) immunoregulatory function, (2) myelin repair, remyelination and/or (3) axonal maintenance/repair. Thus, such a combinatorial regime can comprise the preceding (1-(3) in any order, including concurrent delivery of any of (1)-(3). In some embodiments, a compound that is delivered may confer an immunoregulatory function, as well as enhances myelin repair or remyelination and/or axonal protection. For example, a compound may decrease Th1 or increases Th2 T cell differentiation while concurrently promoting remyelination/myelin repair by enhancing oligodendrocyte regeneration.

In one aspect, an agent delivered to promote myelin repair, remyelination and/or axonal maintenance is a small molecule, chemical compound including pharmaceutical compounds, antisense molecule, aptamer, ribozyme, polypeptide, peptide, peptidomimetic or siRNA. Such a compound targets a cellular process/pathway that is involved in neuronal differentiation, oligodendrocyte differentiation, myelination and/or axonal protection. In one embodiment, an agent inhibits γ-secretase activity/function. In yet other embodiments, one or more agents are administered that affect one or more different cellular pathways associated with neuronal differentiation, oligondendrocyte differentiation and myelination.

Another aspect of the invention is directed to delivering one or more agents that engage a TCR in the absence of the required secondary stimulatory signal, resulting in a TCR signal insufficient to lead to T cell activation (e.g., Th1 or Th2 T cells), whereby anergy, tolerance or cellular depletion results. In some embodiments administration of an immunomodulatory agent(s) is conducted prior to, with, or after delivery one or more additional agents are delivered that promote or enhance remyelination, myelin repair and/or axonal protection.

During MS, as well as during EAE and TMEV-IDD, autoreactive T cell responses directed against myelin and oligodendrocytes (e.g., T-cell responses to proteolipid protein (PLP), myelin basic protein (MBP), myelin oligodendrocyte protein (MOG), or myelin associated oligodendrocytic basic protein (MOBP)) produce inflammatory CNS lesions and neurological dysfunction. Subsequent remyelination may occur to a limited extent that restores neurological function during the early phases of MS. However, continued inflammation and the failure of myelin repair during later stages of disease leads to permanent debilitation. Thus therapeutic strategies disclosed herein include components for immunoregulation, such as suppressing a T-cell activity, differentiation or proliferation and components for promoting or enhancing oligodendrocyte regeneration, myelin repair, remyelination or axonal protection, so as to produce a synergistic therapeutic effect.

In another aspect of the invention, culture or animal models are utilized to screen for synergistic therapeutic effects for treating a neuropathy.

In some embodiments, one or more immunomodulatory agents is administered to a cell or animal model for a neuropathy or related condition, while one or more agents involved in myelin repair/remyelination or axonal protection are administered before, concurrent or subsequent to the immunomodulatory agents, whereby an enhanced or synergistic therapeutic effect, if observed, identify candidate combinatorial treatments. In some embodiments, agents directed to immunomodulation can be small molecules including pharmaceutical compounds, antisense moelcules, siRNA, nucleic acid molecules, peptides, polypeptides, antibodies or aptamers. In other embodiments, agents directed to myelin repair/remyelination or axonal protection can be small molecules including pharmaceutical compounds, antisense, siRNA, nuclei acid molecules, peptides, polypeptides, antibodies or aptamers. In one embodiment, myelin-specific tolerance and myelin repair strategies are two examples of single factor treatment strategies that can be screened.

### SUMMARY OF THE DRAWINGS

FIG. 1 illustrates a model of inflammatory demyelination in EAE and MS. Three generalized therapeutic strategies representing a proposed combinatorial treatment strategy is illustrated.

FIG. 2 illustrates a peptide-coupled peripheral blood leukocyte (PBL) tolerance strategy. Peripheral blood leukocytes (PBLs) are isolated from MS patients and coupled to a cocktail of myelin peptides using ethylene carbodiimide (ECDI)-fixed splenocytes. The antigen-coupled PBLs from an individual pateint is then intravenously reinfused into that patient. The patient is likely to have their long-term tolerance to future autoreactive immune attackes promoted and their immune responses against foreign pathogens not compromised.

FIG. 3 illustrates results underscoring the synergistic benefits of combinatorial treatment.

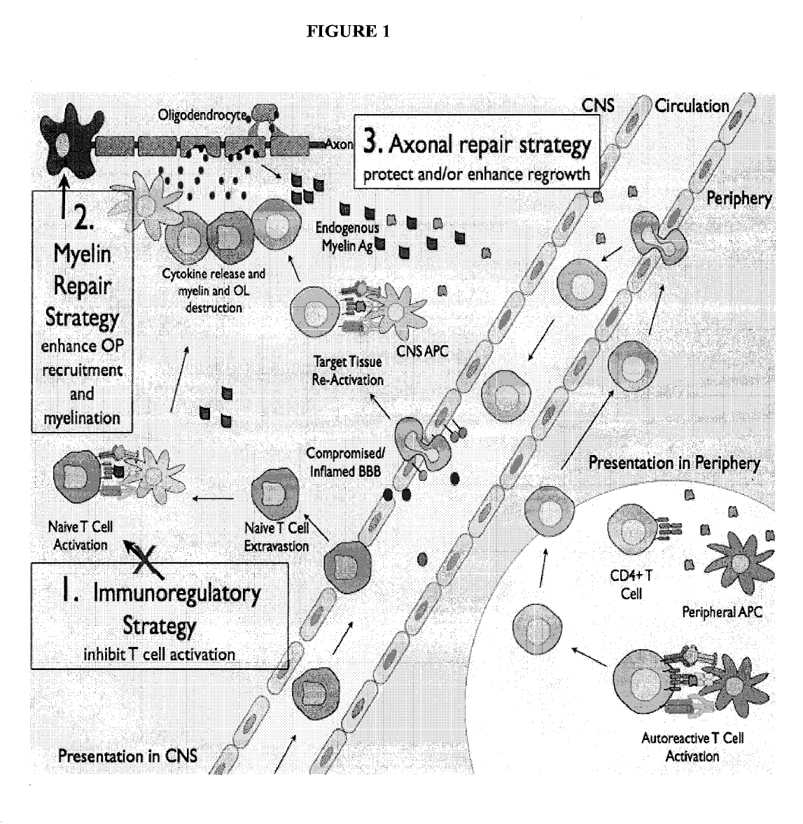

### INCORPORATION BY REFERENCE

All publications and patent applications mentioned in this specification are herein incorporated by reference to the same extent as if each individual publication or patent application was specifically and individually indicated to be incorporated by reference.

### DETAILED DESCRIPTION OF THE INVENTION

### General Techniques:

The practice of the present invention employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, MOLECULAR CLONING: A LABORATORY MANUAL, 2^{nd} edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

### Definitions:

As used in the specification and claims, the singular form “a”, “an” and “the” include plural references unless the context clearly dictates otherwise. For example, the term “a cell” includes a plurality of cells, including mixtures thereof.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

As used herein, “expression” refers to the process by which a polynucleotide is transcribed into mRNA and/or the process by which the transcribed mRNA (also referred to as “transcript”) is subsequently being translated into peptides, polypeptides, or proteins. The transcripts and the encoded polypeptides are collectedly referred to as “gene product.” If the polynucleotide is derived from genomic DNA, expression may include splicing of the mRNA in a eukaryotic cell.

The terms “delivery” and “administration” are used interchangeably herein to mean an agent enters a subject, tissue or cell. The terms used throughout the disclosure herein also include grammatical variances of a particular term. For example, “delivery” includes “delivering”, “delivered”, “deliver”, etc. Various methods of delivery or administration of bioactive agents are known in the art. For example, one or more agents described herein can be delivered parenterally, orally, intraperitoneally, intravenously, intraarterially, transdermally, intramuscularly, liposomally, via local delivery by catheter or stent, subcutaneously, intraadiposally, or intrathecally.

The term “differentially expressed” as applied to nucleotide sequence or polypeptide sequence in a subject, refers to over-expression or under-expression of that sequence when compared to that detected in a control. Under-expression also encompasses absence of expression of a particular sequence as evidenced by the absence of detectable expression in a test subject when compared to a control.

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” refers to either natural and/or unnatural or synthetic amino acids, including glycine and both the D or L optical isomers, and amino acid analogs and peptidomimetics.

A “subject,” “individual” or “patient” is used interchangeably herein, which refers to a vertebrate, preferably a mammal, more preferably a human. Mammals include, but are not limited to mice, rats, dogs, pigs, monkey (simians) humans, farm animals, sport animals, and pets. Tissues, cells and their progeny of a biological entity obtained in vivo or cultured in vitro are also encompassed.

The term “axonal maintenance”, “axonal repair”, “axonal protection” and “axonal regeneration” can be used interchangeably herein.

As used herein “cell” is used in its usual biological sense, and does not refer to an entire multicellular organism. The cell can, for example, be in vitro, e.g., in cell culture, or present in a multicellular organism, including, e.g., birds, plants and mammals such as humans, cows, sheep, apes, monkeys, swine, dogs, cats, mice or rats.

The terms “agent”, “biologically active agent”, “bioactive agent”, “bioactive compound” or “biologically active compound” are used interchangeably and also encompass plural references in the context stated. Such compounds utilized in one or more combinatorial treatment methods of the invention described herein, include, but are not limited to, a biological or chemical compound such as a simple or complex organic or inorganic molecule, peptide, peptide mimetic, protein (e.g. antibody), nucleic acid molecules including DNA, RNA and analogs thereof, carbohydrate-containing molecule, phospholipids, liposome, small interfering RNA, or a polynucleotide (e.g. anti-sense).

The term “control” is an alternative subject, cell or sample used in an experiment for comparison purpose. Furthermore, a “control” can also represent the same subject, cell or sample in an experiment for comparison of different time points.

The term “antibody” as used herein includes all fofrms of antibodies such as recombinant antibodies, humanized antibodies, chimeric antibodies, single chain antibodies, humanized antibodies, fusion proteins, monoclonal antibodies etc. The invention is also applicable to antibody functional fragments that are capable of binding to a therapeutic target (e.g., binding a CD receptor).

The terms “modulating”, “modulated” or “modulation” are used interchangeably and mean a direct or indirect change in a given context. For example, modulation of effector T cell proliferation/stimulation means such proliferation can be modulated downward or upward. In another example, modulation can be of the balance of effector or autoreactive T cells or function/activity thereof, versus regulatory T cells or function/activity thereof.

The term “aptamer” includes DNA, RNA or peptides that are selected based on specific binding properties to a particular molecule. For example, an aptamer(s) can be selected for binding a particular CD using methods known in the art. Subsequently, said aptamer(s) can be administered to a subject to modulate or regulate an immune response. Some aptamers having affinity to a specific protein, DNA, amino acid and nucleotides have been described (e.g., Wang et al., Biochemistry 32:1899-1904 (1993); Pitner et al., U.S. Pat. No. 5,691,145; Gold et al., Ann. Rev. Biochem. 64:763-797 (1995); Szostak et al., U.S. Pat. No. 5,631,146). High affinity and high specificity binding aptamers have been derived from combinatorial libraries (supra, Gold, et al.). Aptamers may have high affinities, with equilibrium dissociation constants ranging from micromolar to sub-nanomolar depending on the selection used. Aptamers may also exhibit high selectivity, for example, showing a thousand fold discrimination between 7-methylG and G (Haller and Samow, Proc. Natl. Acad. Sci. USA 94:8521-8526 (1997)) or between D and L-tryptophan (supra, Gold et al.).

The term “decoy” is meant to include a nucleic acid molecule, for example RNA or DNA, or aptamer, that is designed to preferentially bind to a predetermined ligand or unknown ligand. Such binding can result in the inhibition or activation of a target molecule. The decoy or aptamer can compete with a naturally occurring binding target for the binding of a specific ligand. For example, it has been shown that over-expression of HIV trans-activation response (TAR) RNA can act as a “decoy” and efficiently binds HIV tat protein, thereby preventing it from binding to TAR sequences encoded in the HIV RNA (Sullenger et al., Cell 63, 601-608 (1990)). This is but a specific example and those in the art will recognize that other embodiments can be readily generated using techniques generally known in the art, see for example Gold et al., Annu. Rev. Biochem., 64, 763-797 (1995); Brody and Gold, J. Biotechnol., 74, 5-13 (2000); Sun, Curr. Opin. Mol. Ther., 2, 100-105 (2000); Kusser, J. Biotechnol., 74, 27-38 (2000); Hermann and Patel, Science, 287, 820-825 (2000); and Jayasena, Clinical Chemistry, 45, 1628-1650 (1999). Similarly, a decoy can be designed to bind to a target antigen to occupy its active site, or a decoy can be designed to bind to a target molecule to prevent interaction with another ligand protein(s), thus short-circuiting a cell signaling pathway that is involved in cell proliferation or differentiation.

The term “effective amount” or “therapeutically effective amount” refers to that amount of an agent that is sufficient to effect beneficial or desired results, including without limitation, clinical results such as shrinking the size of demyelinating lesions (in the context of a demyelination disorder, for example), promoting OPC migration, proliferation and growth, delaying the onset of a neuropathy, delaying the development of demyelinating disorder, decreasing symptoms resulting from a neuropathy, increasing the quality of life of those suffering from the disease, decreasing the dose of other medications required to treat the disease, enhancing the effect of another medication such as via targeting and/or internalization, delaying the progression of the disease, decreasing neural scarring, and/or prolonging survival of individuals. The therapeutically effective amount will vary depending upon the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose will vary depending on the particular agent chosen, the dosing regimen to be followed, whether is administered in combination with other compounds, timing of administration, the tissue to be imaged, and the physical delivery system in which it is carried.

The terms “synergistic” or “synergism” mean that a combination of two or more agents when administered as compared to any agent alone results in an enhanced therapeutic effect, whether immunomodulatory, remyelinating or axonal protection/regeneration. In addition, the terms “myelin repair” and “remyelination” (and grammatical nuances of the same) are used interchangeably herein.

### I. Immunomodulation

The present inventions provide methods and compositions for administering agents to effect immunomodulation in a combinatorial process, i.e., with myelin repair, remyelination and/or axonal protection. In preferred embodiments, the combinatorial process provides a synergistic therapeutic effect.

For example, the synergistic effect of two agents may be more than 1 fold than the therapeutic effect of the individual agents alone. In some embodiments, the synergistic effect may have at least 1.2, 1.3, 1.4, or 1.5 fold greater therapeutic effect than either agent alone. In some embodiments, the synergistic effect may have at least 2, 2.2, 2.4, 2.5, 3, 4, 5, 10 or 100 fold greater therapeutic effect than either agent alone. For example, a subject with MS symptoms may be administered a first agent that has no effect on the clinical signs of MS (the mean clinical score of the subject is the same as a subject with MS symptoms not administered the first agent, and both have a mean clinical score of 1.5), but when treated with a second agent, has a mean clinical score of 1, which is a decrease of 0.5. Combination of both agents provides a mean clinical score of 0.4, demonstrating a 1.25 fold synergistic effect (0.5/0.4). In another example, the synergistic effect may be 2, wherein one agent has no effect, for example, a mean clinical score of 1.25 for first agent alone or no agent, and the second agent has a mean clinical score of 0.75, and the combination of both agents has a mean clinical score of 0.25 ([1.25-0.75]/0.25). If both agents have an effect, for example, the first agent has a decrease in mean clinical score of 0.5 (no treatment is 1.5), as does the second agent, treating with both decreases the mean clinical score to 0.1, the two agents have a 5 fold effect ([1.5-0.5-0.5]/0.1]).

The methods disclosed herein can be directed to any neuropathological condition, for example, where degeneration of neural cells occurs or demyelination. Neuronal demyelination is manifested in a large number of hereditary and acquired disorders of the CNS and PNS. Neuropathologies include, but are not limited to, Multiple Sclerosis (MS), Progressive Multifocal Leukoencephalopathy (PML), Encephalomyelitis, Central Pontine Myelolysis (CPM), Anti-MAG Disease, Leukodystrophies, Adrenoleukodystrophy (ALD), Alexander's Disease, Canavan Disease, Krabbe Disease, Metachromatic Leukodystrophy (MLD), Pelizaeus-Merzbacher Disease, Refsum Disease, Cockayne Syndrome, Van der Knapp Syndrome, and Zellweger Syndrome, Guillain-Barre Syndrome (GBS), chronic inflammatory demyelinating polyneuropathy (CIDP), multifocual motor neuropathy (MMN), spinal chord injury (e.g., trauma or severing of), Alzheimer's Disease, Huntington's Disease, Amyotrophic Lateral Sclerosis, Parkinson's Disease, gliosis, astrogliosis and optic neuritis, which have been linked to the degeneration of neural cells in particular locations of the CNS, leading to the inability of neural cells or the brain region to carry out their intended function. In addition, the methods disclosed herein are equally applicable to neuropathy caused by or associated with pathogens including, but not limited to, pathogens causing measles, rabies, scrapie-like agent, Carp agent, paramyxovirus, coronavirus, Epstein-Barr virus, herpes zoster, herpes simplex virus, human herpesvirus 6, rubella, mumps, canine distemper, Marek's Semliki forest virus, animal and human retroviruses, and human T cell lymphoma virus type I.

Immunomodulation with immunomodulatory agents may be by modulating the activity of immune cells. One of skill will recognize that CD4⁺ T cells may typically be the key mediators of the protective immune response through the recognition of pathogens via antigen-specific T cell receptors (TcRs). Thymic selection generates a diverse set of TcRs to ensure protection against foreign pathogens, while negatively selecting against self-specific T cells. Escape from negative selection or deficient peripheral suppressor mechanisms can lead to the breakdown of self tolerance (Christen et al., Curr. Opin. Immunol. 16:759-767 (2004)). Autoreactive CD4⁺ T cells may be directed against myelin antigens including proteolipid protein (PLP), myelin basic protein (MBP), and myelin oligodendrocyte glycoprotein (MOG), and are believed to be involved in MS pathogenesis. Thus, T cells represent a target for therapeutic intervention. In one aspect of the present invention, agents specifically inhibit autoreactive T cells without generalized immunosuppression, for example, without suppressing responses against foreign pathogens.

In one aspect of the invention, a combinatorial treatment process comprises an agent administered to effect immunoregulation/immunomodulation and where said agent is specific to receptors on autoreactive T cells (i.e., effector T cells) to suppress such T cells. An agent may promote a specific response by binding directly or indirectly to the T cells. In some embodiments immunoregulation (also “immunomodulation”) can result in autoreactive T cell depletion, anergy or immune tolerance, or such regulation or modulation can result in altering the balance of autoreactive T cells relative to regulatory T cells (e.g., Th1-Th2 balance). In another aspect, an agent is specific for regulatory T cells and results in differentiation, activation or proliferation of such regulatory T cells.

As disclosed herein, it should be understood that one or more immunomodulation agents as described herein below are administered before, concurrent to, or after administration of one or more agents directed to promoting/enhancing myelin repair, remyelination and/or axonal maintenance/repair, so as to provide an enhanced or synergistic therapeutic effect. In addition, it should be understood that such immunomodulation agents, for any particular endpoint objective, can be a biological or chemical compound such as a simple or complex organic or inorganic molecule, peptide, peptide mimetic, protein (e.g. antibody), carbohydrate-containing molecule, phospholipids, liposome, small interfering RNA, or a polynucleotide (e.g. anti-sense)

A. Cytokine Signaling

Immunomodulatory agents may include agents that modulate the cytokine pathway. Pathway components that may be modulated include, but are not limited to, cytokines and cytokine receptors, chemokine and chemokine receptors, antibodies, complement-related biomarkers, adhesion molecules, antigen processing and/or processing markers, cell cycle and apoptosis-related markers, and agents that may affect their expression or activity. Such factors, receptors and markers are known in the art, and non-limiting examples include, IL-1, IL-2, IL-6, IL-10, IL-12, IL-18, TNF-α, LT-α/γ, TGF-γ, CCR5, CXCR3, CXCL10, CCR2/CCL2, anti-myelin specific protein/peptide antibodies, anti-cluster of differentiation (CD) antibodies, CSF IgG, anti-MOG antibody, anti-MBP antibody, C3, C4, activated neo-C9, regulators of complement activation, E-selectin, L-selectin, ICAM-1, VCAM-1, LFA-1, VLA-4, heat shock proteins, perforin, OX-40, osteopontin, MRP-8 and MRP-16, neopterin, amyloid A protein, somatostatin, Fas, Fas-L, FLIP, Bcl-2, or TRAIL.

Immunomodulatory agents may also modulate the STAT pathway. STAT proteins are a class of molecules that mediate many cytokine-induced responses and are typically activated following phosphorylation via the Janus kinase (JAK) family of tyrosine kinase, which in turn are typically activated via cytokine-cytokine receptor binding. Experimental autoimmune encephalomyelitis (EAE) is generally mediated by myelin-specific CD4(+) T cells secreting Th1 cytokines, while recovery from disease is typically associated with expression of Th2 cytokines. The STAT4 pathway typically controls the differentiation of cells into a Th1 phenotype, while the STAT6 pathway typically controls the differentiation of cells into a Th2 phenotype. In many immune-mediated diseases (autoimmune, allergic, infectious), altering the balance of Th to either Th1 or Th2 correlates to pathogenicity or protection from disease. For example, mice deficient in STAT4 are resistant to the induction of EAE, with minimal inflammatory infiltrates in the central nervous system (e.g., Chitnis et al., J Clin Invest. 108:739-47 (2002)). Furthermore, interaction of IL-4 and the IL-4 cell surface receptor typically results in activation of STAT6, while in a similar interaction IL-12 binds IL-12 receptors to induce Th1 differentiation and IFN-γ production.

In one embodiment, an agent is specific for inhibiting the STAT4 pathway. In another embodiment, an agent is administered to enhance or promote STAT6, resulting in modulation of Th1 versus Th2 balance. In yet another embodiment, an agent is specific for IL-4 receptors. In another embodiment, an agent specific for an IL-4 cell receptor is administered to modulate the Th1 and Th2 balance. In yet another embodiment, an agent specific for an IL-12 receptor is administered to modulate the Th1 and Th2 balance. In another embodiment, an agent that inhibits a JSK activity or function is administered so as to effect modulation of Th1 or Th2 balance. In various embodiments, an agent is a polypeptide, antibody, peptide, aptamer, antisense, siRNA, ribozyme, small molecule, chemical compound, or functional variants thereof. In a preferred embodiment, the various agents that modulate the cytokine pathway are administered before, concurrent, or after administration of one or more agents described herein that promote remyelination, myelin repair or axonal protection.

B. Integrins

Integrins are adhesion molecules that typically confer mechanical stability on interactions between cells and act as cellular sensors and signaling molecules. Integrins are typically composed of noncovalently linked α and β chains. For example, the α₄ integrin chain dimerizes with either the β₁ or β₇ chain. The αβ integrin is also known as CD49d-CD29. By blocking various integrins from binding their respective endothelial counter-receptors, molecular interactions that are required for lymphocytes to enter the central nervous system can be precluded. (Von Adrian and Engelhardt, N. Engl. J. Med. 348: 68-72 (2003)).

In one aspect of the invention, an agent is delivered that is specific for endothelial counter-receptors, thus blocking integrin-receptor interaction, and resulting in modulation of an inflammatory response or level of lymphocyte infiltration of the CNS. In various embodiments, an agent is polypeptide or antibody, peptide, aptamer, antisense molecule, siRNA, ribozyme, small molecule or chemical compound, or functional variants thereof. In one embodiment, an agent is a monoclonal antibody against α₄ integrins. In another embodiment, an agent is an aptamer molecule that is specific for α₄ integrins. In another embodiment, an agent is any small molecule that is specific for α₄ integrins. Various agonists or antagonists of integrins are known in the art, such as disclosed in U.S. Pat. Nos. 6,613,905; 6,602,914; 6,569,996; 7,074,901; 7,074,617; 7,067,525; 7,056,736 and 7,038,018, each of which is incorporated by reference in their entirety. In a preferred embodiment, the various agents that modulate integrins are administered before, concurrent, or after administration of one or more agents described herein that promote remyelination, myelin repair or axonal protection.

C. CD Targeting

Immunomodulatory agents may also target CD molecules, their receptors, and/or their associated proteins. In some embodiments, an agent is administered to effect immunoregulation in one or more combinatorial methods of the invention, wherein said agent is specific for CD3, CD80, CD86 or CD28 ligands or receptors. In some embodiments, the CD protein is CD80, CD28, CD40L, CD3, CD4, CD22, CD25, CD40, CD44, CD45, CD45RB, CD49, CD62, CD69, or CD154. In other aspects of the invention, two or more different agents are delivered wherein each agent is specific for a different CD protein. In other embodiments an agent is specific for STAT4, STAT6, IL-4 or IL-12.

In various embodiments, an agent is a polypeptide, antibody, peptide, aptamer, antisense, siRNA, ribozyme, small molecule or chemical compound, or functional variants thereof, which is specific for CD80, CD86, CD28, CD40L, CD3, CD4, CD22, CD25, CD40, CD44, CD45, CD45RB, CD49, CD62, CD69, CD154 or a combination of two or more thereof.

In some embodiments, an agent is an anti-CD Fab fragment of an antibody, where said Fab fragment is specific for CD80, CD86, CD80-CD86 or CD28. In some embodiments an agentcapable of effecting immunoregulation is specific for CD40L or CD3. In additional embodiments, an agent is specific for CD4, CD22, CD25, CD44, CD45, C45RB, CD49, CD62, CD69, CD154 as well as variants and functional fragments thereof. In yet further embodiments, an agent comprises an Fab fragment that binds any of CD4, CD22, CD25, CD44, CD45, C45RB, CD49, CD62, CD69 or CD154.

In some embodiments, an agent is specific for a CD ligand or its receptor by selectively inhibiting the specific CD signaling pathway. Specific CD signaling may mean that the signal detected results substantially or at least predominantly from the specific CD signaling pathway, and preferably from CD ligand and its receptor interaction, rather than any other significant interfering or competing cause. For example, the agent may be specific for the CD80 signaling pathway, in that the agent substantially affects predominantly the CD80 signaling pathway.

In one aspect of the present invention, an immunomodulatory agent is directed to the CD80-CD28 co-stimulatory pathway for the treatment of a neuropathy. Blockade of co-stimulatory signals represents an attractive strategy to downregulate T cell activation during autoimmune disease. T cell-expressed CD28 typically delivers a critical costimulatory signal when bound by B7 molecules (CD80 and CD86), which are generally found on APCs and activated T cells (Miller et al., Immunol Today. 15:356-361 (1994)). CD80 (B7-1) is typically the dominant costimulatory molecule expressed on CNS infiltrating T-cells in both EAE (Karandikar et al., J. Immunol. 161:192-199 (1998)) and MS (Windhagen et al., J. Exp. Med. 182:1985-1996 (1995)). CD80 can provide regulatory signals for T lymphocytes as a result of CD28 binding CTLA4 ligands of T cells. After engagement of T-cell receptor with antigen in association with major histocompatibility complex class II, a second signal mediated through the binding of B7 to CD28 usually upregulates the production of multiple lymphokines. Blocking the interaction between CD28 and B7 molecules may induce anergy of CD4⁺ T cells (Vanderlugt et al., J. Immunol. 164:670-678 (2000)). Short-term treatment with anti-CD80 Fab fragments during remission in R-EAE can inhibit further relapses by blocking activation of T cells specific for endogenously released myelin epitopes, i.e. epitope spreading (Miller et al., Immunity 3:739-745 (1995)). Conversely, intact anti-CD80 mAb exacerbated ongoing EAE and increased epitope spreading (Miller et al., Immunity 3:739-745 (1995)), likely by cross-linking CD80 on effector T cells leading to IFN-α production and enhanced tissue destruction (Podojil et al., J. Immunol. 177, 2948-2958 (2006)). In other embodiments, the immunoregulatory agent is a CTLA4-Ig fusion protein that antagonizes CD28/B7 costimulation. CTLA4 (CD152) is typically upregulated on activated T cells and acts as a negative regulator of T cell activation following binding of CD80/86 (Bluestone. J. Immunol. 158:1989-1993 (1997)).

In yet another embodiment, CD3 may be targeted by an anti-CD3 antibody. Physically linked with the TcR, the CD3 complex typically functions to transduce activating signals to the T cell upon TcR binding of peptide/MHC complexes on antigen presenting cells (APC). In the absence of secondary costimulatory signals, TcR cross-linking is usually insufficient to activate a T cell and instead induces anergy, tolerance or cellular depletion. In a preferred embodiment, the anti-CD3 antibody is non-mitogenic. Such non-mitogenic anti-CD3 (NM-CD3) mAbs comprise alterations to the Fc region and are known in the art. Furthermore, such antibodies exhibit reduced Fc-receptor binding capacity limiting the ability of the mAbs to cross-link the T cell receptor. Therefore, in some embodiments one or more different anti-CD3 mAbs is administered before, concurrent or after an agent is administered to enhance remyelination, myelin repair or axon protection, wherein the combination can produce an enhanced or synergistic therapeutic effect.

In another aspect of the invention, antibodies are administered to a subject to effect immunomodulation by targeting B cells. In some embodiments an antibody is specific to a B-cell antigen, including but limited to CD22, CD20, CD19, and CD74 or HLA-DR antigen. The antibodies are administered alone or in combination, and may be naked or conjugated to a drug, toxin or therapeutic radioisotope. Bispecific antibody fusion proteins which bind to the B-cell antigens can be used according to the present invention, including hybrid antibodies which bind to more than one B-cell antigen.

In preferred embodiments, the various agents that modulate CDs are administered before, concurrent, or after administration of one or more agents described herein that promote remyelination, myelin repair or axonal protection.

D. Altered Peptide Ligands (APLs)

Immunomodulatory agents may provide antigen-specific targeting of the autoreactive T cells for the treatment of autoimmune disease, in combination with agents effecting myelin repair, remyelination and/or axonal protection. APLs are variant peptides of autoantigens typically substituted at the TcR contact residues to lower TcR signaling affinity and elicit different functional responses (Sloan-Lancaster, Annu. Rev. Immunol. 14:1-27 (1996)). In vivo administration of various myelin epitope APLs have successfully prevented and/or ameliorated ongoing clinical disease progression in EAE (Samson et al., J. Immunol. 155:2737-2746 (1995)). The mechanistic actions of APLs include the induction of T cell anergy (Illes et al., Proc. Natl. Acad. Sci. U.S.A. 101:11749-11754 (2004)), Th1 (pro-inflammatory) to Th2 (anti-inflammatory) cytokine switch (Fischer et al., J. Neuroimmunol. 110:195-208 (2000)), and bystander immune suppression by regulatory T cells (Nicholson. Proc. Natl. Acad. Sci. U.S.A. 94:9279-9284 (1997)); Bielekova et al., Nat. Med. 6:1167-1175 (2000)). In various embodiments, the variants can include altered peptides derived from anti-APC, myelin basic protein (MBP), ceramide galactosyltransferase (CGT), myelin associated glycoprotein (MAG), myelin oligodendrocyte glycoprotein (MOG), oligodendrocyte-myelin glycoprotein (OMG), cyclic nucleotide phosphodiesterase (CNP), NOGO, myelin protein zero (MPZ), peripheral myelin protein 22 (PMP22), protein 2 (P2), galactocerebroside (GalC), sulfatide and proteolipid protein (PLP).

In some embodiments, MHC-anchored-substituted peptides are utilized to effect immunomodulation in a combinatorial treatment process that also promotes remyelination, myelin repair and/or axonal protection/maintenance. In some embodiments, variant peptides are for MOG₃₅₋₅₅ (amino acids 35-55; subscripts refer to amino acid sequence numbers) (Ford et al., J. Immunol. 171:1247-1254 (2003)) and PLP₁₃₉-₁₅₁ (Margot et al., J. Immunol. 174:3352-3358 (2005)) peptides, containing an amino acid substitution at the MHC anchor residue, thereby typically affecting polyclonal T cell populations and eliminating the activation of cross-reactive T cells. The MOG₃₅₋₅₅ APL has been shown to induce anergy in multiple MOG₃₅₋₅₅-specific T cell and polyclonal lines and reduced the ability of MOG₃₅₋₅₅-specific T cells to transfer EAE in an adoptive transfer model (Ford et al., J. Immunol. 171:1247-1254 2003)). The PLP₁₃₉₋₁₅₁ APL has been shown to decrease severity of established R-EAE (Margot et al., J. Immunol. 174:3352-3358 (2005)). In some embodiments, the APL may be NBI-5788. In some embodiments, MHC-anchored-substituted peptides are administered before, concurrent, or after administration of one or more agents described herein that promote remyelination, myelin repair or axonal protection

In some embodiments, lower dosages for a variant peptide can affect therapeutic results, including reduced MRI lesions and induction of Th2 responses. This latter effect is similar to, without being bound by theory, the mechanistic properties of an approved drug for the treatment of relapsing-remitting MS, glatiramer acetate (GA; copolymer 1; copaxone). Initially shown to block progression of EAE, GA is a random copolymer of amino acids (YEAK) designed to mimic MBP (Sanna et al., Clin. Exp. Immunol. 143:357-362 (2006)). GA is reported to selectively compete with activation of MBP-specific autoreactive T cells and induce MBP-specific Th2-regulatory cells. GA therapy leads to a modest reduction in clinical relapses in some, but not all, MS patients, which onsets approximately six months following the onset of therapy. Modified copolymers of GA (VWAK and FYAK) also affect T-cell responses (Illes et al., Proc. Natl. Acad. Sci USA 101: 11749-11754 (2004)).

In a preferred embodiment, variant peptides are administered before, concurrent, or after administration of one or more agents described herein that promote remyelination, myelin repair or axonal protection.

E. Peptide-Coupled Cell Tolerance

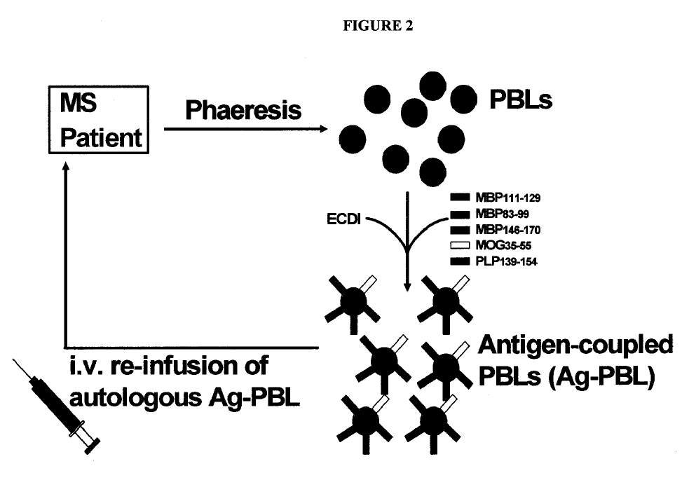

In one aspect, immunomodulation, such as induction of T cell tolerance, is effected by delivering ethylene carbodiimide (ECDI)-fixed splenocytes by intravenous injection of myelin peptide-pulsed, ECDI-fixed splenocytes either prior to or following disease onset. (Miller et al., Immunol. Rev. 144:225-244 (1995)). Epitope spreading in R-EAE typically follows a hierarchical order, with PLP₁₃₉₋₁₅₁ being the dominant encephalitogenic epitope and PLP₁₇₈₋₁₉₁ and MBP₈₄₋₁₀₄ following sequentially (Vanderlugt et al., J. Immunol. 164:670-678 2000)). Tolerance can be induced in R-EAE by injecting splenocytes coupled to either the priming peptide (to block onset of disease), the spread epitopes (to block specific relapses), or a combination of myelin peptides (Vanderlugt et al., J. Immunol. 164:670-678 (2000)). This tolerance protocol can successfully ameliorate ongoing EAE and appears to induce T cell anergy, via both direct and indirect pathways, and activates regulatory T cells. In sum, this technique appears to be an efficient and safe process for restoring antigen-specific tolerance and will s be tested in a phase I clinical trial in relapsing-remitting MS patients (FIG. 2).

Immunomodulation through antigen-specific tolerance is attractive given its potential to suppress autoimmunity without compromising protective immune responses. Peptide-coupled cell tolerance can be promising as it is an effective therapy for ongoing autoimmune disease with no obvious side effects.

In one aspect, agents are delivered to effect immunomodulation are multi-peptide-coupled-cells to induce tolerance (FIG. 2). In various embodiments, the immunomodulatory peptides coupled to cells include MBP₁₃₋₂₂, MBP₁₁₁₋₁₂₉, MBP₁₅₄₋₁₇₀, PLP₁₃₉₋₁₅₄, MOG₁₋₂₀ and/or MOG₃₅₋₅₅. Bielekova et al., J. Immunol. 172:3893-3904 (2004)). In other embodiments, the immunomodulatory peptides include MBP₈₃₋₉₉, MBP₁₄₆₋₁₇₀, MBP₁₃₁₋₁₅₅, PLP₄₀₋₆₀, PLP₈₉₋₁₀₆, PLP₁₇₈₋₁₉₇, MOG₁₁₋₃₀, CNP₃₄₃₋₃₇₃, and/or CNP₃₅₆₋₃₈₈. In a preferred embodiments, the various peptide-coupled cells are administered before, concurrent, or after administration of one or more agents described herein that promote remyclination, myelin repair or axonal protection.

F. DNA Vaccination

In some aspects of the present invention, immunomodulatory agents are DNA vaccines. DNA vaccines have been used as a means to generate protective immunity in several autoimmune models. Vaccinations with DNA encoding various encephalitogenic myelin peptides or proteins have been shown to be protective against EAE development in various rat and mouse models (Fountoura et al., Int. Rev. Immunol. 24:415-446 (2005)). In some embodiments, animals are co-vaccinated with multiple myelin DNA constructs with Th2-type constructs, such as IL-4. Animals may be vaccinated with constructs encoding MOG, PLP, MBP, and/or MAG. In some embodiments, the animals may additionally be vaccinated with an IL-4 vaccine (Robinson et al., Nat. Biotechnol. 21:1033-1039 (2003)). In other embodiments, GpG oligodeoxynucleotide (ODN), with a cytosine-guanine base switch, may be used in combination with one or more of the DNA vaccines described herein (Ho et al., J. Immunol. 175:6226-6234 (2005); Ho et al., J. Immunol. 171:4920-4926 (2003)). The DNA vaccine may be BHT-3009. In a preferred embodiment, the DNA vaccines are administered before, concurrent, or after administration of one or more agents described herein that promote remyelination, myelin repair or axonal protection.

G. T Cell Receptor Vaccination

In another aspect of the present invention, pathogenic Th1 cells may be modulated by T cell receptor (TCR) vaccines. As expression of the same, or similar, TCR variable (V) genes is common among T cells that respond to a specific autoantigen, the set of TcR V regions on the α and β chains (AV and BV) may be used to derive peptides for TCR vaccines (Vandenbark et al., Crit. Rev. Immunol. 20:57-83 (2000)). For example, TCR peptides may correspond to the AV2 or BV8S2 genes. TCR BV genes BV5 and BV6 are commonly expressed genes of MBP-specific T cells in the blood and cerebrospinal fluid (CSF) and brains of MS patents (Kotzin et al., Proc. Natl. Acad. Sci USA 88:9161-9165 (1991); Wilson et al., J. Neuroimmunol. 76:15-28 (1997); Olsenberg et al., Nature 362:68-70 (1993)), and vaccines may be derived from these genes. Overexpression of BV13S1 in MBP-specific T cells has also been identified (Vandenbark et al., Nat. Med. 2:1109-1115 (1996)), and can also be used to derive TCR vaccines. Vaccines with peptides derived from BV5S2, BV5S2 with a substitution (Y49T), and the trivalent TCR peptide vaccine IR902 (Neurovax™), a combination of BV5S2, BV6S5, and BV13S1 in incomplete Freud's adjuvant (IFA), may also be used as immunomodulatory agents (Vandenbark et al., Nat. Med. 2:1109-1115 (1996); Gold et al., J. Neuroimmunol. 76:29-38 (1997)). In a preferred embodiment, the T cell receptor vaccines are administered before, concurrent, or after administration of one or more agents described herein that promote remyelination, myelin repair or axonal protection.

### II. MYELIN REPAIR STRATEGIES

The immunoregulatory components discussed herein can impede future immune attacks against myelin, but have not been shown, with the possible exception of CTLA4-Ig (Neville et al., J. Virol. 74:8349-8357 (2000)), to promote myelin repair. While remyelination occurs early during MS, the repair of most CNS lesions is generally not achieved. The failure of myelin repair, coupled with progressive clinical debilitation, justifies a need for additional pharmacological intervention to promote remyelination and protect axons from further damage. Enhancement of endogenous remyelination and transplantation of cells with myelinogenic potential are two general approaches for remyelinating therapies. The present invention provides compositions and method for treating demyelinating conditions by administering an immunomodulatory agent with remyelinating therapies. In some embodiments, the remyelinating promoting agent is administered concurrent with, subsequent to, or prior to an immunomodulatory agent. In some embodiments, a synergistic therapeutic effect is achieved. In some embodiments, an axonal promoting agent is also administered, concurrent with, subsequent to, or prior to the immunomodulatory and myelin repair promoting agents. The myelin repair agent my promote proliferation, migration or differentiation of oligodendrocytes. In some embodiments, the immunomodulatory agent is administered concurrent with, subsequent to, or prior to an agent that promotes oligodendrocyte differentiation. In yet another embodiment, an oligodendrocyte proliferation promoting agent is further administered to a subject or cell, concurrent with, subsequent to, or prior to the agents that promote oligodendrocyte differentiation and immunomodulation.

A. Endogenous Remyelination

To repair the damage of repeated immunological attacks, sufficient numbers of oligodendrocytes (OLs) must be replaced and these cells must efficiently contact and remyelinate denuded axons. It is well established that oligodendrocyte progenitor (OP) cells, and not OLs surviving demyelination episodes, are responsible for remyelination (Dawson et al., Mol. Cell. Neurosci. 24:476-488 (2003), Watanabe et al., J. Neurosci. Res. 69:826-836 (2002), Keirstead et al., J. Neuropathol. Exp. Neurol. 56:1191-1201 (1997), Gensert et al., Neuron. 19:197-203 (1997)). Thus, it is likely that the failure of remyelination is associated with deficiencies in the proliferation, migration, and/or differentiation of adult OP cells. Developmental studies have extensively characterized the process of myelination and identified a myriad of factors and signaling pathways involved in the regulation of OL generation and myelin wrapping.

B. Growth Factors

In some aspects of the invention the myelin repair/remyelination component of the combinatorial methods of the invention are agents that are cellular growth factors. Growth factors such as, but are not limited to, nerve growth factor (NGF), Brain-Derived neurotrophic factor (BDNF), neutrotrophin-3 (NT-3) and ciliary neurotropic factor (CNTF) may be used in the present invention. NGF (total dose infused i.v.=1 ug) has been reported to ameliorate cholinergic neuron atrophy and spatial memory impairment in aged rats by W. Fischer et al., Nature 329:65-68 (1987). Recombinant human β NGF has been produced which has potent in vitro and in vivo neurotropic activity. See J. Barrett et al., Exp. Neurol. 110:11-24 (1990). In some embodiments, the nerve growth factors are administered exogenously, such as in polypeptide pharmaceutical formulations. In other embodiments, the nerve growth factors can be delivered by transfection of target cells in vivo or in vitro followed by transplantation into target sites.

In other embodiments, myelin promoting agents include but are not limited to, neurotrophins, cytokines of the neuropoietin family, and neuregulins (Aloisi. Neurol. Sci. 24 Suppl 5:S291-294 (2003)). The agents may also include, but not limited to, biological molecules which have been shown to influence the processes of oligodendrocyte survival, proliferation, migration and differentiation, such as Platelet Derived Growth Factor (PDGF) (Jean et al., Neuroreport 13:627-631 (2002)), Thyroid Hormone (TH) (Calza et al., Proc. Natl. Acad. Sci. USA 99:3258-3263 (2002)), Granulocyte Colony Stimulating Factor (GCSF) (Zavala et al., J. Immunol. 168:2011-2019. (2002)), Ciliary Neurotrophic Factor (CNTF) (Linker et al., Nat. Med. 8:620-624 (2002)), Fibroblast Growth Factor-2 (FGF-2) (Armstrong et al., J. Neurosci. 22:8574-8585 (2002)), Leukemia Inhibitory Factor (LIF) (Butzkueven et al., Nat. Med. 8:613-619 (2002)), Insulin Like Growth Factor-1 (IGF-1) (Beck et al., Neuron 14:717-730 (1995)), Glial Growth Factor-2/Neuregulin (GGF-2/NRG) (Kerber et al., J. Mol. Neurosci. 21:149-165 (2003)) and CXCL1/Growth Regulated Oncogene Alpha (Gro-α) (Omari et al., Glia 53:24-31 (2006); Omari et al., Brain 128:1003-1015 (2005); Tsai et al., Cell 110:373-383 (2002)).

Some factors have been tested in clinical trials (Frank et al., Mult. Scler. 8:24-29 (2002); Villoslada et al., J. Exp. Med. 191:1799-1806 (2000); Althaus. Prog. Brain Res. 146:415-432 (2004)). Cytokines and chemokines have previously been shown to influence OP cell fate decisions (French-Constant et al., Trends Cell Biol. 14:678-686 (2004), Agresti et al., Eur. J. Neurosci. 8:1106-1116 (1996), Ambrosini et al., Neurochem. Res. 29:1017-1038 (2004)). In some embodiments a synergistic therapeutic result can be enhanced by administering a plurality of growth factors to induce remyelination. (Scolding et al., Neuroreport 6:441-445 (1995), Chandran et al., Glia. 47:314-324 (2004)). In some embodiments, growth factors can be administered which include neuroregulin glial growth factor 2 or thyroid hormone to effect oligodendrocyte progenitor maturation and remyelination.

C. Human Monoclonal Antibodies/Intravenous Immunoglobulins

Intravenous administration of immunoglobulins (IVIg) was originally developed for the treatment of antibody deficiencies, but has since been used for treating various autoimmune and systemic inflammatory conditions (Trebst et al., Curr. Pharm. Des. 12:241-249 (2006), Humle et al., J. Neurol. Sci. 233:61-65, (2005)). IVIg consists of mainly IgG molecules with a diversity of specificities prepared from pooled plasma of numerous healthy donors. IVIg can downregulate the immune system through various mechanisms including suppression of autoantibodies via anti-idiotypic interactions, modulation of macrophage and T cell finction, and inhibition of cytokine production (Trebst et al., Curr. Pharm. Des. 12:241-249 (2006), Humle et al., J. Neurol. Sci. 233:61-65 (2005)). IVIg has been utilized in EAE (Jorgensen et al., Neurol. Res. 27:591-597 (2005)), as well as clinical trials (Lewanska et al., Eur. J. Neurol. 9:565-572 (2002), Sorensen et al., Neurology 50:1273-1281 (1998), Sorensen. J. Neurol. Sci. 206:123-130 (2003), Hommes et al., Lancet 364:1149-1156 (2004)), Trebst et al., Curr. Pharm. Des. 12:241-249 (2006), Humle et al., J. Neurol. Sci. 233:61-65 (2005)).

In one embodiment, an IgM antibody is administered to a subject to promote myelin repair or remyelination. In some embodiments, the antibodies are monoclonal IgM antibodies that can bind to oligodendrocytes and promote intracellular signaling, so as to promote remyelination in vivo (Warrington et al., Proc. Natl. Acad. Sci. U.S.A. 97:6820-6825 (2000), Warrington et al., J. Allergy Clin. Immunol. 108:S121-125 (2001)). In one embodiment, a monoclonal IgM antibody, such as rHIgM22 (also known as rsHIgM22, sHIgM22 and LYM 22), is administered to promote remyelination.(Ciric et al., J. Neuroimmunol. 146:153-161 (2004); Howe et al., Neurobiol. Dis. 15:120-131 (2004); US Publication No. 20070086999). Other human antibodies that may be administered include ebvHIgM, MS119D10, sHIgM46 (LYM46), ebvHIgMCB2b-G8, or MS110E10 (US Publication No. 20070086999). In some embodiments, the antibody is administered prior to, subsequent to, or concurrent with an immunomodulatory agent.

D. γ-secretase Inhibition

In some aspects an agent that promotes myelin repair or remyelination modulates γ-secretase activity. In some embodiments, an agent that promotes myelin repair or remyelination modulates Notch signaling (Jurynczyk et al., J. Neuro. Sci. PMID: 1794975 (2007)). In preferred embodiments, the agent that modulates γ-secretase activity and/or Notch signaling is administered prior to, subsequent to, or concurrent with an immunomodulatory agent.

Without being limited to any particular mechanism, epitope spreading typically occurs in the CNS (McMahon et al., Nat. Med. 11:335-339 (2005)) and inhibition of γ-secretase and/or Notch signaling can inhibit T cell responses specific for the spread epitope. However, expression of Notch1 on OLs after CNS demyelination is not inhibitory or rate-limiting for remyelination (Stidworthy et al., Brain. 127:1928-1941 (2004)).

Notch proteins (Notch1-4) are transmembrane glycoprotein receptors that interact with at least six identified ligands including Jagged 1 and 2, Delta-like 1, 3, and 4, and contactin. Upon ligand binding, Notch is typically cleaved by the enzyme γ-secretase and the intracellular domain translocates to the nucleus where it acts as a transcription factor regulating a host of cellular processes including the inhibition of neuronal differentiation (Yoon et al. Nat. Neurosci. 8:709-715 (2005)), oligodendrocyte differentiation, and myelination (Givogri et al., J. Neurosci. Res. 67:309-320 (2002), Wang et al., Neuron. 21:63-75 (1998), Genoud et al., J. Cell Biol. 158:709-718 (2002)). Notch 1 and Jagged1 are usually expressed on immature OLs and hypertrophic astrocytes, respectively, within MS plaques that lack remyelination (John et al., Nat. Med. 8:1115-1121 (2002)). Notch signaling has also been implicated in the regulation of mature T cell function. Previously described functions of Notch include tolerance induction (Hoyne et al., Int. Immunol. 12:177-185 (2000)), T regulatory cell differentiation (Hoyne et al., Int. Immunol. 12:177-185 (2000); Yvon et al., Blood. 102:3815-3821 (2003); Vigouroux et al., J. Virol. 77:10872-10880 (2003)), and promotion (Adler et al., J. Immunol 171:2896-2903 (2003); Palaga et al., J. Immunol. 171:3019-3024 (2003)) or inhibition (Benson et al., Eur. J. Immunol. 35:859-869 (2005); Eagar et al., Immunity. 20:407-415 (2004)) of T cell proliferation and cytokine production (Adler et al., J. Immunol. 171:2896-2903 (2003); Palaga et al., J. Immunol. 171:3019-3024 (2003); Benson et al., Eur. J. Immunol. 35:859-869 (2005); Eagar et al., Immunity. 20:407-415 (2004)).

In various embodiments, agents are administered that are specific for Notch 1, Notch 2, Notch 3, Notch 4, Jagged 1, Jagged 2, Delta-like 1, Delta-like 2, Delta-like 3 or contactin. In other embodiments, the agents are specific for γ-secretase and can inhibit γ-secretase activity or function. In other embodiments, agents bind Notch 1, Notch 2, Notch 3, Notch 4, Jagged 1, Jagged 2, Delta-like 1, Delta-like 2, Delta-like 3 or contactin, or a combination of two or more thereof, which agents are administered so as to short circuit Notch signaling thereby interfering with inhibition of neuronal differentiation, oligodendrocyte differentiation and myelination (i.e., promoting myelin repair or remyelination). In other embodiments, an agent regulates Notch signaling indirectly, for example inhibiting downstream effects of the Notch pathway. In some embodiments, an agent specific to Notch 1, Notch 2, Notch 3, Notch 4, Jagged 1, Jagged 2, Delta-like 1, Delta-like 2, Delta-like 3 or contactin is an aptamer, peptide, peptidomimetic or antibody. In other embodiments, an agent of the present invention is a γ-secretase inhibitor. Such agents are known in the art (Benson et al., Eur. J. Immunol. 35:859-869 (2005), Eagar et al.,Immunity. 20:407-415 (2004), Minter et al., Nat. Immunol. 6:680-688 (2005); US Publ. No. 20070225228). Inhibitors of γ-secretase may include N-[N-(3,5-Difluorophenacetyl-L-alanyl]-S-phenylglycine-t-butyl ester (DAPT), which inhibits both PS-1 and PS-2. This compound is an optimized derivative of a molecule that inhibited Aβ production in a screen of approximately 25,000 compounds. DAPT is a cell-permeable dipeptide non-transition state analog that can compete moderately for the γ-secretase active site in a displacement assay, suggesting some overlap between the binding site of DAPT and the active site. Other examples of β-secretase inhibitors include: Compound III-31-C, Compound E, Isocoumarins, D-Helical peptide 294, Epoxide, (Z-LL)₂-ketone (a SPP inhibitor) (see US Publication No. 20070225228). Other γ-secretase inhibitors include peptidomimetic inhibitors such as L-685,458 ((5S)-(t-Butoxycarbonylamino)-6-phenyl-(4R)hydroxy-(2R)benzylhexanoyl)-L-leu-L-phe-amide), described by Shearmen et al., Biochemistry 39:8698-8704 (2000). Another inhibitor of γ-secretase, described by Wolfe et al., J. Med. Chem. 41:6 (1998), is ALX-260-127 (also referred to as compound 11), which is a reversible difluoro ketone peptidomimetic inhibitor. Other inhibitors include photoactivated γ-secretase inhibitors directed to the active site of γ-secretase, for example, as described by Li et al., Nature 405(6787):689-94 (2000), and sulindac sulfide (SSide), which directly acts on δ-secretase and preferentially inhibits the γ-secretase activity in an in vitro γ-secretase assay using recombinant amyloid beta precursor protein C100 as a substrate, as described in Takahashi et al., J. Biol. Chem. 278:18664-70 (2003). γ-secretase inhibitors may also include, but not be limited to those, such as 5-(Arylsulfonyl)pyrazolopiperidines, bridged N-cyclic sulfonamide compounds, bridged N-bicyclic sulfonamides, dibenzoazepine, transmembrane cargo protein TMP21, benzenesulfonyl-chromane, thiochromane, tetrahydronaphthalene, tetrahydroindoles, fluoro substituted 2-oxo-azepan derivatives, cycloalkyl, lactam, lactone and related compounds, N-(aryl/heteroaryl/alkylacetyl) amino acid amides, trifluoromethyl-containing phenylsulfonamide, heterocyclic sulfonamide derivatives, fluoro- and trifluoroalkyl-containing heterocyclic sulfonamides, substituted phenylsulfonamide derivatives, for example, as described in PCT Publ. Nos. WO2007064914, WO2007022502, WO2007110667, WO2007084595, WO2007054739, WO2007024651, WO199828268, WO9822433, WO2003103660, US Publ. Nos. US 2007225273, US 2007037789, US2007249722, US2005171180, US2004198778, Canadian Pat. Appl. CA2581109. The □-secretase inhibitor may also be LY450139 (FIG. 4).

Recent studies showed that the specific γ-secretase inhibitor, LY411575, had no effect on T cell proliferation, but decreased Th1 differentiation in vitro, and lessened the severity of EAE when injected prior to disease onset (Minter et al., Nat. Immunol. 6:680-688 (2005)) and that intraventricular administration of the inhibitor, MW167, following the onset of clinical disease enhanced tissue repair and recovery from acute EAE (Jurynczyk et al., J. Neuroimmunol. 170:3-10 (2005)). Preferred embodiments for γ-secretase inhibitors include LY411575, III-31-C or DAPT. In yet other embodiments, agents inhibiting γ-secretase include antisense molecules, siRNA, aptamers, peptides, polypeptides, other small molecules or antibodies.

To inhibit γ-secretase activity, genetic agents that directly inhibit the expression of presenilin, e.g. anti-sense oligonucleotides that hybridize to a portion of the presenilin transcript; and the like, may be introduced. Such methods also encompass the use of interference RNA (RNAi) technology. In this approach, a molecule of double-stranded RNA specific to a subunit γ-secretase, e.g. presenilin, is used. RNAi technology refers to a process in which double-stranded RNA is introduced into cells, e.g. oligodendrocytes, expressing a subunit of γ-secretase to inhibit expression of the targeted gene, i.e., to “silence” its expression. The dsRNA is selected to have substantial identity with the targeted gene. In general such methods initially involve in vitro transcription of a nucleic acid molecule containing all or part of a targeted gene sequence into single-stranded RNAs. Both sense and anti-sense RNA strands are allowed to anneal under appropriate conditions to form dsRNA. The dsRNA is prepared to be substantially identical to at least a segment of a targeted gene. The resulting dsRNA is introduced into cells via various methods, thereby silencing expression of the targeted gene. Because only substantial sequence similarity between the targeted gene and the dsRNA is necessary, sequence variations between these two species arising from genetic mutations, evolutionary divergence and polymorphisms can be tolerated. Moreover, the dsRNA can include various modified or nucleotide analogs. Usually the dsRNA consists of two separate complementary RNA strands. However, in some instances, the dsRNA may be formed by a single strand of RNA that is self-complementary, such that the strand loops back upon itself to form a hairpin loop. Regardless of form, RNA duplex formation can occur inside or outside of a cell. A number of established gene therapy techniques can also be utilized to introduce the dsRNA into a cell. By introducing a viral construct within a viral particle, for instance, one can achieve efficient introduction of an expression construct into the cell and transcription of the RNA encoded by the construct.

The methods of the present invention also includes inhibiting γ-secretase by expression of dominant-negative or familial Alzheimer's disease (FAD) mutants of presenilin-1 or presenilin-2 and the knockout/disruption of genes (or gene products) that are essential for γ-secretase activity, such as presenilin, nicastrin, Pen-2, or Aph-1.

E. Transplantation of Remyelinating Cells

In another aspect of the invention, an agent administered to promote myelin repair or remyelination is a cell that affects myelination. In some embodiments the cells are oligodendrocyte progenitor cells (OPC), Schwann cells (SCs), olfactory bulb ensheathing cells, and neural stem cells (NSCs), which are administered prior to, concurrent with or subsequent to one or more immunomodulatory agents. In one embodiment, such cells are cultured and expanded in vitro prior to transplantation. In various embodiments, the cells may be transfected or genetically modified in vitro or in vivo to express or express at modified levels a polypeptide that effects immunomodulation and/or myelin repair or axonal protection. In some embodiments, the myelin producing cells or progenitor cells thereof include but are not limited to fetal or adult OPCs. In one embodiment the OPC may be A2B5⁺PSA⁻NCAM⁻ phenotype (positive for the early oligodendrocyte marker A2B5 and negative for polysialylated neural cell adhesion molecule).

Remyelination of CNS axons has been demonstrated in various animal models (Stangel et al., Prog. Neurobiol. 68:361-376 (2002); Pluchino et al., J. Neurol. Sci. 233:117-119 (2005)). Many recent studies have since demonstrated new techniques and novel mechanisms associated with the use of cell transplantation in demyelinating disease. Human OP cells isolated from adult brains were able to myelinate naked axons when transplanted into a dysmyelinating mouse mutant (Windrem et al., Nat. Med. 10:93-97 (2004)). The use of adult progenitor cells may avoid ethical concerns. While OP cells are typically responsible for endogenous remyelination, NSCs are an alternative source of cells to promote myelin repair. NSCs are found in the adult CNS, can be expanded extensively in vitro, and can differentiate to form OLs, astrocytes, or neurons. When transplanted into rodents with relapsing or chronic forms of EAE, NSCs have been shown to migrate to areas of CNS inflammation and demyelination and to preferentially adopt a glial cell-fate (Ben-Hur et al., Glia. 41:73-80 (2003), Pluchino et al., Nature 436:266-271 (2005), Pluchino et al., Nature 422:688-694 (2003), Einstein et al. Exp. Neurol. 198:129-135 (2006)). Attenuation of clinical disease in transplanted mice was associated with repair of demyelinating lesions and decreased axonal injury (Pluchino et al., Nature 436:266-271 (2005), Pluchino et al., Nature 422;688-694 (2003), Einstein et al., Exp. Neurol. 198:129-135 (2006)). Histological analysis confirmed that transplanted NSCs differentiated predominantly into PDGFR⁺ OP cells (Pluchino et al., Nature. 422:688-694 (2003)).

Interestingly, while the number of OP cells was increased in NSC-transplanted EAE mice, the majority of these cells were not donor-derived, suggesting that the transplanted cells regulated the expansion of endogenous oligodendroglia (Pluchino et al., Nature. 422:688-694 (2003)). The mechanisms by which NSCs promote EAE amelioration and lesion repair are indicative of immunosuppressive and neuroprotective functions. NSCs have been demonstrated to induce apoptosis of T cells both in vivo and in vitro (Pluchino et al. Nature. 436:266-271 (2005)), to decrease CNS infiltrating T cells in NSC-transplanted EAE rodents and to inhibit myelin peptide-specific T cell proliferation in vitro (Einstein et al., Exp. Neurol. 198:129-135 (2006), Einstein et al., Mol. Cell. Neurosci. 24:1074-1082 (2003)). The immunomodulatory and proposed neuroprotective properties may be mediated by neurotrophic factors (Lu P et al., Exp. Neurol. 181:115-129 (2003)) and various growth factors (Einstein et al., Exp. Neurol. 198:129-135 (2006)) which may decrease CNS inflammation and/or enhance OL lineage cell survival and promote remyelination in the host CNS.

In some embodiments, oligodendrocyte progenitor cells (OPC), Schwann cells (SCs), olfactory bulb ensheathing cells, and neural stem cells (NSCs) are transfected with one or more expression vectors, which are described herein above, so as to enable expression of one or more desired agent. Such agents can be directed to the immunomodulation, myelin repair/remyelination or axonal protection. In various embodiments, the cells are transfected before, concurrent or subsequent to expansion in culture.

It will be appreciated that transplantation is conducted using methods known in the art, including invasive, surgical, minimally invasive and non-surgical procedures. Depending on the subject, target sites, and agent(s) to be the delivered, the type and number of cells can be selected as desired using methods known in the art. The transplantation may be performed prior to, subsequent to, or concurrent with administration of another agent, such as an immunomodulatory agent.

### III. AXONAL PROTECTION

In another aspect, in combination with immunomodulatory or myelin repair agents, agents that promote axonal protection or regeneration can also be administered to produce a synergistic therapeutic effect. In some embodiments, agents the block inhibitory axonal regeneration signals are administered to a subject or cell. In various embodiments, such bioactive agents can be antisense probes, siRNA, aptamers, peptides, polypeptides, other small molecules or antibodies. For example, an antibody can bind Nogo and short circuit the axonal regeneration inhibition.

Nogo is a member of the reticulon family, expressed by oligodendrocytes but not by Schwann cells and inhibits axonal extension. The Nogo receptor complex, composed of the Nogo-66 receptor 1, neurotrophin p75 receptor and LINGO-1, represses axon regeneration upon binding to myelin-associated inhibitory factors. The binding of neurotropin to its receptor, p75 neurotrophic tyrosinekinase receptor, abolishes activation of protein kinase C and the GTPase ras homolog gene family member A and decreases neurite outgrowth. (Yamashita et al., Neuron. 24:585-593 (1999)). Antibodies against Nogo-66 protect against EAE while some Nogo-66 epitopes induce protective Th2 cell lines. (Frontoura et al., J. Immunol. 173:6981-6992 (2004)).

Injured oligodendrocytes and myelin exert negative signals for axonal re-generation. Calpains that are found in glia and inflammatory cells can degrade myelin proteins at physiological pH. As a result, neuronal self-repair and axonal regeneration may be impaired by signals released during myelin destruction. Among the products of myelinolysis, myelin-associated glycoprotein and Nogo inhibit axonal regeneration and are collectively called myelin-associated inhibitory factors.

Therefore, in some embodiments therapeutic targets to simulate axonal regeneration include inhibitors of Nogo signaling and protein kinase C inhibitors. In one embodiment, antibodies are administered that are specific for Nogo-66. In other embodiments, peptides, aptamers, antisense or siRNA target any member of the Nogo receptor complex, whereby binding preclude Nogo signaling thus obviating inhibition of axonal regeneration. In other embodiments, a bioactive agent is specific for neurotropin, neurotropin p75 receptor or LINGO-1.

In preferred embodiments, the agent promoting axonal protection is administered prior to, subsequent to, or concurrent with an immunomodulatory agent.

### IV. AGENTS

Immunomodulatory, myelin repair promoting, and axonal protection promoting agents as described herein include, without being limited to, peptides, polypeptides, antisense molecules, aptamers, siRNAs, external guide sequence (EGS) small organic molecules, antibodies, peptidomimetics, or vaccines. These agents can be provided in linear or cyclized form, and optionally comprise at least one amino acid residue that is not commonly found in nature or at least one amide isostere. These compounds may be modified by glycosylation, phosphorylation, sulfation, lipidation or other processes.

Agents may encompass numerous chemical classes, including organic molecules, organometallic molecules, inorganic molecules, and genetic sequences. Agents include organic molecules comprising functional groups necessary for structural interactions, particularly hydrogen bonding, and typically include at least an amine, carbonyl, hydroxyl or carboxyl group, frequently at least two of the functional chemical groups. Agents may comprise cyclical carbon or heterocyclic structures and/or aromatic or polyaromatic structures substituted with one or more of the above functional groups. Agents are also found among biomolecules, including peptides, polynucleotides, saccharides, fatty acids, steroids, purines, pyrimidines, derivatives, structural analogs or combinations thereof. Included are pharmacologically active drugs and genetically active molecules. Agents may also include chemotherapeutic agents, and hormones or hormone antagonists. Pharmaceutical agents may also be suitable for this invention, such as described in, “The Pharmacological Basis of Therapeutics,” Goodman and Gilman, McGraw-Hill, New York, N.Y., (1996), Ninth edition. Agents may be obtained from a wide variety of sources including libraries of synthetic or natural compounds, for example compounds identified in screening assays as described below.

Agents may also include antibodies, such as anti-CD80 or anti-CD3 antibodies. Producing such antibodies as described herein are known in the art, such as disclosed in U.S. Pat. Nos. 6,491,916; 6,982,321; 5,585,097; 5,846,534; 6,966,424 and U.S. Patent Application Publication Nos. 20050054832; 20040006216; 20030108548, 2006002921 and 20040166099, each relevant portion of which is incorporated herein by reference. In merely one example, monoclonal antibodies can be obtained by injecting mice with a composition comprising the antigen, verifying the presence of antibody production by removing a serum sample, removing the spleen to obtain B-lymphocytes, fusing the B-lymphocytes with myeloma cells to produce hybridomas, cloning the hybridomas, selecting positive clones which produce antibodies to the antigen that was injected, culturing the clones that produce antibodies to the antigen, and isolating the antibodies from the hybridoma cultures. Monoclonal antibodies can be isolated and purified from hybridoma cultures by a variety of well-established techniques. Such isolation techniques include affinity chromatography with Protein-A Sepharose, size-exclusion chromatography, and ion-exchange chromatography. See, for example, Coligan at pages 2.7.1 2.7.12 and pages 2.9.1 2.9.3. Also, see Baines et al., “Purification of Immunoglobulin G (IgG),” in METHODS IN MOLECULAR BIOLOGY, VOL. 10, pages 79 104 (The Humana Press, Inc. 1992).

Suitable amounts of well-characterized antigen for production of antibodies can be obtained using standard techniques. As an example, CD antigen proteins can be obtained from transfected cultured cells that overproduce the antigen of interest. Expression vectors that comprise DNA molecules encoding each of these proteins can be constructed using published nucleotide sequences. See, for example, Wilson et al., J. Exp. Med. 173:137-146 (1991); Wilson et al., J. Immunol. 150:5013-5024 (1993). As an illustration, DNA molecules encoding CD3 can be obtained by synthesizing DNA molecules using mutually priming long oligonucleotides. See, for example, Ausubel et al., (eds.), CURRENT PROTOCOLS IN MOLECULAR BIOLOGY, pages 8.2.8 to 8.2.13 (1990). Also, see Wosnick et al., Gene 60:115-127 (1987); and Ausubel et al. (eds.), SHORT PROTOCOLS IN MOLECULAR BIOLOGY, 3rd Edition, pages 8-8 to 8-9 (John Wiley & Sons, Inc. 1995). Established techniques using the polymerase chain reaction provide the ability to synthesize genes as large as 1.8 kilobases in length. (Adang et al., Plant Molec. Biol. 21:1131-1145 (1993); Bambot et al., PCR Methods and Applications 2:266-271 (1993); Dillon et al., “Use of the Polymerase Chain Reaction for the Rapid Construction of Synthetic Genes,” in METHODS IN MOLECULAR BIOLOGY, Vol. 15: PCR PROTOCOLS: CURRENT METHODS AND APPLICATIONS, White (ed.), pages 263 268, (Humana Press, Inc. 1993)). In a variation, monoclonal antibody can be obtained by fusing myeloma cells with spleen cells from mice immunized with a murine pre-B cell line stably transfected with cDNA which encodes the antigen of interest. (See Tedder et al., U.S. Pat. No. 5,484,892.)

In one embodiment, an entire, naked antibody or combination of entire, unlabeled antibodies are immunomodulatory agents. In some embodiments, antibody fragments are utilized, thus less than the complete antibody. In other embodiments, conjugates of antibodies with drugs, toxins or therapeutic radioisotopes are useful. Bispecific antibody fusion proteins which bind to the CD antigens can be used according to the present invention, including hybrid antibodies which bind to more than one antigen. Preferably the bispecific and hybrid antibodies additionally target a T-cell, plasma cell or macrophage antigen. Therefore, antibody encompasses naked antibodies and conjugated antibodies and antibody fragments, which may be monospecific or multispecific.

Depending on the characteristics of the agent, an agent can be delivered via plasmid vectors, viral vectors or non-viral vector systems, including liposome formulations and minicells. Therefore in some embodiments an agent, such as a myelin repair promoting nerve growth factor, is encoded by a nucleic acid sequence that is transfected into a target cell. Therefore, the desired growth factor is expressed from the nucleic acid sequence which can be integrated into the cell genome, or present on a plasmid or viral vector. In some embodiments, the one or more agents co-administered to effect immunomodulation, myelin repair/remyelination or axonal protection is expressed from a nucleic acid sequence. In some embodiments, the expression of a nucleic acid sequence encoding such an agent is inducible thus temporal.

Agents of the present invention may directly or indirectly modulate the activity levels of T cell activation, preferably autoreactive T cells, and/or γ-secretase activity. In some embodiments, glial cells are cultured and transfected with expression constructs in vitro and subsequently administered to a subject, wherein the expression constructs encode agents such as inhibitors of γ-secretase. Therefore, in some embodiments, modulated expression is effected through ex vivo methods.

In some embodiments, nucleic acids encoding an agent that modulates the immune response can be co-administered with nucleic acids encoding an agent that promotes remyelination in a combinatorial fashion. For example, two or more co-administered agents expressed from the nucleic acid may promote migration, proliferation, and/or differentiation of glial cells, as well as inhibit or reduce autoimmune responses. The agent expressed from the nucleic acid may block or inhibit autoimmune responses, for example, by inhibiting autoreactive T cells. Agents that suppress autoimmune responses can be combined with agents promoting myelin repair, such as through inhibiting γ-secretase activity. In some further embodiments, the expression of a nucleic acid sequence encoding such an agent is inducible thus temporally controlled. Such inducible or temporally controlled transcription regulatory elements are known in the art and as further disclosed herein. Genetically modifying or transfecting cells either in vitro or in vivo can be conducted utilizing methods known in the art, as described in references noted herein, and such as disclosed in U.S. Pat. Nos. 6,998,118; 6,670,147 or 6,465,246.

In other embodiments, such transfected cells include SCs, NSCs, OPCs, astrocytes, microglial cells or a combination of such cells, which can be transfected in culture or in vivo. In some embodiments, the expression constructs comprise cell-specific or inducible promoters, which are specific for glial cells, and are described herein, as well as known to one of ordinary skill in the art.

Examples of neural cells used in one or more methods of the invention include glial cells, such as oligodendrocytes, oligodendrocyte progenitors, Schwann cells, astrocytes, and microglia. Within the microenvironment of the CNS, astrocytes provide support and nourishment, oligodendrocytes provide insulation, and microglia provide immune defense. Astrocytes, commonly identified by the expression of the intermediate filament protein glial fibrillary acidic protein (GFAP), possess a variety of ion channels, transporters, and neurotransmitter receptors that help maintain brain homeostasis and may alter neuronal excitability. In addition, astrocytes interact with endothelial cells, and these interactions are thought to be critical for the development and maintenance of the blood brain barrier (BBB). Astrocytes are known to react to CNS injury by proliferating, changing their morphology, expanding processes, and enhancing their expression of GFAP. This activation, termed astrocytosis or astrogliosis, may lead to deposition of extracellular matrix molecules (ECM) into a dense fibrous scar. Such a response to injury is considered detrimental for repair. Furthermore, following injury, astrocytes can activate glutamate receptors leading to excitotoxicity and death of surrounding cells.

Neural cells of the present invention also includes oligodendrocytes, which are the macroglial cells typically responsible for the production and maintenance of CNS myelin, the fatty insulation that enwraps axons to enhance the speed and reliability with which information is transmitted. Oligodendrocytes typically first develop in the CNS from the ventral ventricular and subventricular zones of the spinal cord and brain. Oligodendrocytes in the spinal cord typically arise from the ventricular zone during embryonic development and subsequently migrate to white matter where they proliferate and differentiate (Miller, Prog. Neurobiol. 67:451-467 (2002)). During their maturation and differentiation, oligodendrocytes typically go through a sequence of developmental stages characterized by distinct alterations in cell morphology and the expression of specific molecular markers. The specificity of these markers for individual cell populations allows identification of cells—at different stages and opportunities for their isolation.

In some embodiments, glia cells are microglia, which as the name suggests, are the smallest of the three CNS glial cells and share characteristics with bone marrow derived monocytes and macrophages to which they are related. They are derived from myeloid progenitor cells of lymphoid tissues and are thought to arrive to the CNS during its developmental vascularization. Resting microglia have elongated bipolar cell bodies with perpendicular spine-like processes. Microglia are highly motile cells and, when activated, are thought to act like immune cells in the CNS, with phagocytosis, presentation of antigens, and secretion of inflammatory cytokines. Astrocytes and microglia may act as antigen presenting cells and that this behavior may amplify immune responses and lead to uncontrolled myelin destruction.

Expression of an agent from an expression vector may be placed under the control of one or more regulatory elements, such as constitutive or inducible promoters, tissue-specific regulatory elements, and enhancers. Such an agent is said to be “operably linked to” the regulatory elements. For example, constitutive, inducible or cell/tissue specific promoters can be incorporated into an expression vector to regulate expression of a nucleic acid sequence that is expressed in a host cell.

In some embodiments, an agent which is expressed from a nucleic acid sequence can be operably linked to one or more transcription regulatory sequences that are specific to neural cells. Exemplary transcriptional regulatory sequences/elements include transcriptional regulatory sequences/elements selected from the genes encoding the following proteins: the PDGFα receptor, proteolipid protein (PLP), the glial fibrillary acidic gene (GFAP), myelin basic protein (MBP), neuron specific enolase (NSE), oligodendrocyte specific protein (OSP), myelin oligodendrocyte glycoprotein (MOG) and microtubule-associated protein 1B (MAP1B), Thy1.2, CC1, ceramide galactosyltransferase (CGT), myelin associated glycoprotein (MAG), oligodendrocyte-myelin glycoprotein (OMG), cyclic nucleotide phosphodiesterase (CNP), NOGO, myelin protein zero (MPZ), peripheral myelin protein 22 (PMP22), protein 2 (P2), tyrosine hydroxylase, BSF1, dopamine 3-hydroxylase, Serotonin 2 receptor, choline acetyltransferase, galactocerebroside (GalC), and sulfatide. Furthermore, examples of neural cell-specific promoters are known in the art, such as disclosed in U.S. Patent Application Publication No. 2003/0110524; See also, the website <chinook.uoregon.edu/promoters.html>. Additionally, cell/tissue specific promoters are also known in the art.

In some embodiments, the transcriptional regulatory elements are inducible. For example, non-limiting examples of inducible promoters include metallothionine promoters and mouse mammary tumor virus promoters. Other examples of promoters and enhancers effective for use in the recombinant vectors of the present invention include, but are not limited to, CMV (cytomegalovirus), SV40 (simian virus 40), HSV (herpes simplex virus), EBV (Epstein-Barr virus), retrovirus, adenoviral promoters and enhancers, and smooth-muscle-specific promoters and enhancers; strong constitutive promoters that may be suitable for use as the heterologous promoter include the adenovirus major later promoter, the cytomegalovirus immediate early promoter, the β-actin promoter, or the β-globin promoter. Promoters activated by RNA polymerase III could also be used.

In some embodiments, inducible promoters that have been used to control gene expression include the tetracycline operons, RU 486, heavy metal ion inducible promoters such as the metallothionein promoter; steroid hormone inducible promoters, such as the MMTV promoter, or the growth hormone promoter. Promoters which would be inducible by the helper virus such as adenovirus early gene promoter inducible by adenovirus E1A protein, or the adenovirus major late promoter; herpesvirus promoter inducible by herpesvirus proteins such as VP16 or 1CP4; vaccinia or poxvirus inducible promoters or promoters inducible by a poxvirus RNA polymerase; bacterial promoter such as that from T7 phage which would be inducible by a poxvirus RNA polymerase; or a bacterial promoter such as that from T7 RNA polymerase, or ecdysone, may also be used. In one embodiment, a promoter element is a hypoxic response elements (HRE) recognized by a hypoxia-inducible factor-1 (HIF-1) which is one of the key mammalian transcription factors that exhibit dramatic increases in both protein stability and intrinsic transcriptional potency during low-oxygen stress. HRE has been reported in the 5′ or 3′ flanking regions of VEGF and Epo and several other genes. The core consensus sequence is (A/G)CGT(G/C)C. HREs isolated from Epo and VEGF genes have been used to regulate several genes, such as suicide gene and apoptosis gene expression in hypoxic tumors to enhance tumor killing.

Furthermore, where expression of the transgene in particular subcellular location is desired, the transgene can be operably linked to the corresponding subcellular localization sequences by recombinant DNA techniques widely practiced in the art. Exemplary subcellular localization sequences include but are not limited to (a) a signal sequence that directs secretion of the gene product outside of the cell; (b) a membrane anchorage domain that allows attachment of the protein to the plasma membrane or other membraneous compartment of the cell; (c) a nuclear localization sequence that mediates the translocation of the encoded protein to the nucleus; (d) an endoplasmic reticulum retention sequence (e.g. KDEL sequence) that confines the encoded protein primarily to the ER; (e) proteins can be designed to be farnesylated so as to associate the protein with cell membranes; or (f) any other sequences that play a role in differential subcellular distribution of a encoded protein product.

Vectors utilized in in vivo or in vitro methods can include derivatives of SV-40, adenovirus, retrovirus-derived DNA sequences and shuttle vectors derived from combinations of finctional mammalian vectors and functional plasmids and phage DNA. Eukaryotic expression vectors are well known, e.g. such as those described by Southern and Berg, J. Mol. Appl. Genet. 1:327-341 (1982); Subramini et al., Mol. Cell. Biol. 1:854-864 (1981), Kaufinann and Sharp, J. Mol. Biol. 159:601-621 (1982); Scahill et al., Proc. Natl. Acad. Sci. USA 80:4654-4659 (1983) and Urlaub and Chasin, Proc. Natl. Acad. Sci. USA 77:4216-4220 (1980), which are hereby incorporated by reference. The vector used in the methods of the present invention may be a viral vector, preferably a retroviral vector. Replication deficient adenoviruses are preferred. For example, a “single gene vector” in which the structural genes of a retrovirus are replaced by a single gene of interest, under the control of the viral regulatory sequences contained in the long terminal repeat, may be used, e.g. Moloney murine leukemia virus (MoMulV), the Harvey murine sarcoma virus (HaMuSV), murine mammary tumor virus (MuMTV) and the murine myeloproliferative sarcoma virus (MuMPSV), and avian retroviruses such as reticuloendotheliosis virus (Rev) and Rous Sarcoma Virus (RSV), as described by Eglitis and Andersen, BioTechniques 6:608-614 (1988), which is hereby incorporated by reference.

Recombinant retroviral vectors into which multiple genes may be introduced may also be used according to the methods of the present invention. Vectors with internal promoters containing a cDNA under the regulation of an independent promoter, e.g. SAX vector derived from N2 vector with a selectable marker (neo^{R}) into which the cDNA for human adenosine deaminase (hADA) has been inserted with its own regulatory sequences, the early promoter from SV40 virus (SV40), may be designed and used in accordance with the methods of the present invention by methods known in the art.

In mammalian host cells, a number of viral-based expression systems can be utilized. In cases where an adenovirus is used as an expression vector, the nucleotide sequence of interest (e.g., encoding a therapeutic capable agent) can be ligated to an adenovirus transcription or translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) may result in a recombinant virus that is viable and capable of expressing the gene product in infected hosts. (See e.g., Logan & Shenk, Proc. Natl. Acad. Sci. USA 8 1:3655-3659 (1984)).

Specific initiation signals can also be required for efficient translation of inserted therapeutic nucleotide sequences. These signals include the ATG initiation codon and adjacent sequences. In cases where an entire therapeutic gene or cDNA, including its own initiation codon and adjacent sequences, is inserted into the appropriate expression vector, no additional translational control signals may be needed. However, in cases where only a portion of the therapeutic coding sequence is inserted, exogenous translational control signals, including, perhaps, the ATG initiation codon, may be provided. Furthermore, the initiation codon may be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (See e.g., Bittner et al., Methods in Enzymol, 153:516-544 (1987)).

### V. SCREENING ASSAYS

A. Cell Culture

The present invention also provides methods of screening different combinations of immunomodulatory and myelin repair or axonal protection inducing agents to determine which combination is beneficial in treating a neuropathy. Combinations may provide a synergistic therapeutic effect.

In some embodiments, neural cells, particularly glial cells, more particularly, astrocytes, oligodendrocytes, SCs, OPCs or NSCs are cultured and/or genetically modified and used for screening. In some embodiments of the invention, a co-culture system (see US Publication No. 20070225228) may be useful for examining crucial axon-glial interactions that regulate myelination distinct from factors that simply influence the differentiation of purified OPCs, and can be used for screening assays. Acutely-purified neurons, e.g. retinal ganglion cells, dorsal root ganglion cells, etc., can be plated at high density on a non-adhesive substrate for a period of time sufficient for reaggregation, usually from about one, two three or more days. During this time, the neurons adhere to one another in reaggregates of tens to hundreds of cells. These reaggregates may then be collected and plated on protein, e.g. laminin, etc. coated coverslips, after which they typically rapidly extend dense beds of axons radially. Few dendrites typically extend from these reaggregates. Under these conditions, neuronal cell bodies and dendrites are spatially restricted, creating multiple regions of dense axon beds. Acutely-purified oligodendrocyte progenitor cells (OPC) are added after a period of time sufficient for axon formation, usually about one week. After addition of the OPC, myelin segments can be observed by MBP immunostaining or electron microscopy within as little as seven days in culture. Although the co-culture is permissive for myelination, the majority of MBP-expressing OLs will typically still fail to myelinate the many adjacent axons.

The culture may contain growth factors to which the cells are responsive. Growth factors, as defmed herein, are molecules capable of promoting survival, growth and/or differentiation of cells, either in culture or in the intact tissue, through specific effects on a transmembrane receptor. Growth factors include polypeptides and non-polypeptide factors. The specific culture conditions are chosen to achieve a particular purpose, i.e. maintenance of progenitor cell activity, etc. In some embodiments of the invention, the co-cultures are grown in the absence of trophic factors that are conventionally used to support their long-term survival of neurons and oligodendrocytes in culture. Typical cultures contain, in addition to other factors, CNTF and forskolin. In the cultures of the present invention, the trophic support between neuron and oligodendrocyte provide sufficient factors to allow the removal of these exogenously added trophic factors, thus minimizing interfering effects of exogenous factors.

The subject co-cultured cells may be used in a variety of ways. For example, the nutrient medium, which is a conditioned medium, may be isolated at various stages and the components analyzed. Separation can be achieved with HPLC, reversed phase-HPLC, gel electrophoresis, isoelectric focusing, dialysis, or other non-degradative techniques, which allow for separation by molecular weight, molecular volume, charge, combinations thereof, or the like. One or more of these techniques may be combined to enrich further for specific fractions that promote myelination.

In one embodiment, one or more immunonodulatory agent(s) is placed in contact with such a culture of cells, and before, concurrent or subsequent to such contact, one or more myelin repair- or axonal protection-inducing agent is also administered to the cells, to determine which immunomodulatory agent and myelin repair- or axonal protection-inducing agent produces a desired effect, preferably, a synergistic effect. For example, the combination of immunomodulatory agent and myelin repair- or axonal protection-inducing agent has a greater effect in promoting remyelination as compared to cells treated with the immunomodulatory agent alone or cells treated with the myelin repair- or axonal protection-inducing agent.

In another embodiment, one or more immunonodulatory agent(s) is placed in contact with such a culture of cells, and before, concurrent or subsequent to such contact, one or more myelin repair-inducing agent is also administered to the cells. A third agent, such as an axonal protection promoting agent may then be administered before, concurrent or subsequent to the previous two agents, and the effect of all three agents may be determined to identify which immunomodulatory, myelin repair-inducing, and axonal protection-inducing agents produce a desired effect, preferably, a synergistic effect. For example, the combination of immunomodulatory agent and myelin repair- or axonal protection-inducing agent has a greater effect in promoting remyelination and axonal protection as compared to cells treated with the agents alone or with two of the three agents.

In yet another embodiment, one or more immunonodulatory agent(s) is placed in contact with such a culture of cells, and before, concurrent or subsequent to such contact, one or more oligodendrocyte differentiation promoting agent is also administered to the cells. A third agent that promotes oligodendrocyte proliferation or migration may then also be administered before, concurrent or subsequent to the previous two agents, and the effect of all three agents may be determined to identify which immunomodulatory, oligodendrocyte proliferation/migration and differentiation-inducing agents produce a desired effect, preferably, a synergistic effect. For example, the combination the agents has a greater effect in promoting remyelination as compared to cells treated with the agents alone or with two of the three agents.

A synergistic effect may be observed in culture by utilizing time-lapse microscopy revealing a transition from precursor cell types to myelinating oligodendrocyte. Furthermore, progenitor cells can be transfected with a membraned-targeted form of enhanced green fluorescent protein (EGFP) to facilitate convenient fluorescence microscopy in detection of differentiated cells. Therefore, in various embodiments, cells can be cultured and/or genetically modified to express marker proteins or immunomodulatory, myelin repair-promoting, or axonal protection-promoting agents that are components of a combinatorial treatment or screening process utilizing techniques that are known in the art, such as disclosed in U.S. Pat. Nos. 7,008,634; 6,972,195; 6,982,168; 6,962,980; 6,902,881; 6,855,504; or 6,846,625.

In one embodiment, an expression vector can encode a marker protein (e.g., fluorescent marker) that is expressed from a cell-specific promoter element (e.g., PLP or PDGFα, which are specific for glial cells, including oligodendrocytes). Further, the same cells can be transfected with a second expression vector that encodes an immunomodulatory agent, such as an expression vector that encodes an APL. Alternatively, a single expression construct can encode more than one polypeptide, such as marker protein and an APL. In other embodiments, more than one immunomodulatory agent or myelin-repair promoting agent may be expressed by one or more expression vectors, for example, vectors encoding an APL and siRNA for PS-1. Cells expressing one or more immunomodulatory agents and/or myelin-repair promoting agents and one or more marker proteins can be detected using standard microscopy techniques known in the art, including but not limited to fluorescence microscopy (including for example, in vitro cell or tissue culture or in vivo imaging).

In some embodiments, neural cells are transfected with a nucleic acid molecule that is operably linked to a constitutive, inducible or neural-cell-specific promoter and encodes an immunomodulatory or myelin-repair promoting agent. Such cells can be transformed to express the one or more agents at altered levels. Furthermore, such cells can be administered to an animal subject to modulate the immune response and promote remyelination. In one embodiment, cells are genetically modified to provide an altered T-cell response. In preferred embodiments, the altered T-cell response is combined with promotion of remyelination. In one embodiment, neural cells are genetically modified to express APLs. Nucleic acids encoding a desired APL and/or γ-secretase inhibitor can be transformed into target cells by homologous recombination, integration or by utilization of plasmid or viral vectors utilizing components and methods described herein and familiar to those of ordinary skill in the art.

In should be clear to one of ordinary skill in the art, that expression levels in neural cells can be altered by expression of a desired polypeptide encoded on an expression construct that is administered to such cells. Alternatively, expression can be modulated by utilizing expression constructs that encode a product (e.g., antisense molecule, siRNA, aptamer) that itself affects expression of a desired polypeptide. Antisense molecules, siRNA or aptamers can be selected utilizing processes familiar to one of skill in the art. Other agents, such as antibodies and small molecules, such as those described above, may also alter the expression or activity of proteins involved in a signaling pathway, such as the Notch pathway, activation of T cells, or epitope spreading. These pathways may be affected by altering the expression of components involved in the pathways, or expression of its upstream regulators or ligands, or its downstream effectors. In some embodiments, screening assays are performed for agents that act synergistically with γ-secretase inhibitors, for example immunomodulatory agents, that promote remyelination can be performed. Immunomodulatory agents can be screened for an effect on the inhibition of myelination, e.g. by adding a candidate agent to the culture system in the presence of a γ-secretase inhibitor. Addition of a γ-secretase inhibitor can strongly increase the number of myelin segments detected by MBP and MOG staining. Myelin segments can be observed in as little as three days after plating acutely-purified OPCs, with a large number of myelinating OLs observed by six days in culture. Normal paranodal and nodal differentiation can also be observed in these cultures by immunostaining. In screening assays for biologically active agents, cells, usually cocultures of cells (as described herein) are contacted with an agent of interest, and the effect of an agent assessed by monitoring output parameters, such as extent of myelination, expression of markers, cell viability, and the like. Various assays have also been described for screening γ-secretase inhibitors, for example by Takahashi et al., J Biol. Chem. 278:18664-70 (2003), an assay based on detection of the putative C-terminal fragment-γ of APP by Pinnix et al., J Biol. Chem. 276:481-487 (2002); cell free assays for γ-secretase activity by McLendon et al., FASEB J 14:2383-2386 (2000).

Other cellular parameters may be quantified to determine the effect of the agents. Parameters are quantifiable components of cells, particularly components that can be accurately measured, desirably in a high throughput system. A parameter can be any cell component or cell product including cell surface determinant, receptor, protein or conformational or posttranslational modification thereof, lipid, carbohydrate, organic or inorganic molecule, nucleic acid, e.g. MRNA, DNA, etc. or a portion derived from such a cell component or combinations thereof. While most parameters will provide a quantitative readout, in some instances a semi-quantitative or qualitative result will be acceptable. Readouts may include a single determined value, or may include mean, median value or the variance, etc. Characteristically a range of parameter readout values will be obtained for each parameter from a multiplicity of the same assays. Variability may be expected and a range of values for each of the set of test parameters shall be obtained using standard statistical methods with a common statistical method used to provide single values.

Agents of interest for screening include known and unknown compounds that encompass numerous chemical classes, primarily organic molecules, which may include organometallic molecules, inorganic molecules, genetic sequences, etc. An important aspect of the invention is to evaluate candidate drugs, including toxicity testing; and the like.

Candidate agents include organic molecules comprising functional groups necessary for structural interactions, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof. Included are pharmacologically active drugs, genetically active molecules, etc. Compounds of interest include chemotherapeutic agents, hormones or hormone antagonists, etc. Exemplary of pharmaceutical agents suitable for this invention are those described in, “The Pharmacological Basis of Therapeutics,” Goodman and Gilman, McGraw-Hill, New York, N.Y., (1996), Ninth edition. Also included are toxins, and biological and chemical warfare agents, for example see Somani, S. M. (Ed.), “Chemical Warfare Agents,” Academic Press, New York, 1992).

Compounds, including candidate agents, are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fuingal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Agents can be screened for biological activity by adding an agent to at least one and usually a plurality of cell samples, usually in conjunction with cells lacking the agent. In other embodiments, the candidate agent is added to cells treated with a first agent and compared to cells treated with the first agent alone, and/or candidate agent alone. The candidate agent may be added to the cells prior to the first agent, concurrent with the first agent, or subsequent to the first agent. For example, cells may be treated with a first agent such as anti-CD80(Fab). The cells treated with anti-CD80(Fab) are contacted with candidate agents prior to, concurrent with, or subsequent to the cells contact with anti-CD80(Fab). Candidate agents are selected based on their ability or promote remyelination to a greater effect as compared to cells treated with anti-CD80(Fab) alone or the candidate agent alone. Alternatively, cells may be treated with a first agent that is a γ-secretase inhibitor such as DAPT or LY411575. Candidate agents that act synergistically with the γ-secretase inhibitor may be selected for further analysis. More than two agents may be screened, for example, in the aforementioned embodiments, a third agent can be screened and compared to the cells treated with just two agents, to determine if there is a synergistic effect with the third agent.

The change in parameters in response to an agent is measured, and the result evaluated by comparison to reference cultures, e.g. in the presence and absence of the agent, obtained with other agents, etc. In preferred embodiments, the agents selected after screening confer a synergistic effect. The agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of an agent or at or below the concentration of agent that does not give a detectable change in the phenotype.

Markers may be parameters used to detect the effect of the candidate agents and combination of agents. Various methods can be utilized for quantifying the presence of the selected markers. For measuring the amount of a molecule that is present, a convenient method is to label a molecule with a detectable moiety, which may be fluorescent, luminescent, radioactive, enzymatically active, etc., particularly a molecule specific for binding to the parameter with high affinity. Fluorescent moieties are readily available for labeling virtually any biomolecule, structure, or cell type. Immunofluorescent moieties can be directed to bind not only to specific proteins but also specific conformations, cleavage products, or site modifications like phosphorylation. Individual peptides and proteins can be engineered to autofluoresce, e.g. by expressing them as green fluorescent protein chimeras inside cells (for a review, see Jones et al., Trends Biotechnol. 17:477-81 (1999)).

Detection of the gene expression level for markers can be conducted in real time in an amplification assay. In one aspect, the amplified products can be directly visualized with fluorescent DNA-binding agents including but not limited to DNA intercalators and DNA groove binders. Because the amount of the intercalators incorporated into the double-stranded DNA molecules is typically proportional to the amount of the amplified DNA products, one can conveniently determine the amount of the amplified products by quantifying the fluorescence of the intercalated dye using conventional optical systems in the art. DNA-binding dye suitable for this application include SYBR green, SYBR blue, DAPI, propidium iodine, Hoechste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, and the like.

In another aspect, other fluorescent labels such as sequence specific probes can be employed in the amplification reaction to facilitate the detection and quantification of the amplified products. Probe-based quantitative amplification relies on the sequence-specific detection of a desired amplified product. It utilizes fluorescent, target-specific probes (e.g., TaqMan probes) resulting in increased specificity and sensitivity. Methods for performing probe-based quantitative amplification are well established in the art and are taught in U.S. Pat. No. 5,210,015.

In yet another aspect, conventional hybridization assays using hybridization probes that share sequence homology with marker genes can be performed. Typically, probes are allowed to form stable complexes with the target polynucleotides contained within the biological sample derived from the test subject in a hybridization reaction. It will be appreciated by one of skill in the art that where antisense is used as the probe nucleic acid, the target polynucleotides provided in the sample are chosen to be complementary to sequences of the antisense nucleic acids. Conversely, where the nucleotide probe is a sense nucleic acid, the target polynucleotide is selected to be complementary to sequences of the sense nucleic acid.

As is known to one skilled in the art, hybridization can be performed under conditions of various stringency. Suitable hybridization conditions for the practice of the present invention are such that the recognition interaction between the probe and target is both sufficiently specific and sufficiently stable. Conditions that increase the stringency of a hybridization reaction are widely known and published in the art. See, for example, (Sambrook, et al., (1989), supra; Nonradioactive In Situ Hybridization Application Manual, Boehringer Mannheim, second edition). The hybridization assay can be formed using probes immobilized on any solid support, including but are not limited to nitrocellulose, glass, silicon, and a variety of gene arrays. A hybridization assay is conducted on high-density gene chips as described in U.S. Pat. No. 5,445,934.

For a convenient detection of the probe-target complexes formed during the hybridization assay, the nucleotide probes are conjugated to a detectable label. Detectable labels suitable for use in the present invention include any composition detectable by photochemical, biochemical, spectroscopic, immunochemical, electrical, optical or chemical means. A wide variety of appropriate detectable labels are known in the art, which include fluorescent or chemiluminescent labels, radioactive isotope labels, enzymatic or other ligands. In various embodiments, one may likely desire to employ a fluorescent label or an enzyme tag, such as digoxigenin, β-galactosidase, urease, alkaline phosphatase or peroxidase, avidin/biotin complex.

The detection methods used to detect or quantify the hybridization intensity will typically depend upon the label selected above. For example, radiolabels may be detected using photographic film or a phosphoimager. Fluorescent markers may be detected and quantified using a photodetector to detect emitted light. Enzymatic labels are typically detected by providing the enzyme with a substrate and measuring the reaction product produced by the action of the enzyme on the substrate; and finally colorimetric labels are detected by simply visualizing the colored label.

An agent-induced change in gene expression or an agent-induced effect can also be determined by examining the corresponding gene products. Determining the protein level typically involves a) contacting the protein contained in a biological sample comprising myelinating cells with an agent that specifically bind to the protein being detected; and (b) identifying any agent:protein complex so formed. In one aspect of this embodiment, an agent that specifically binds a CD is an antibody, preferably a monoclonal antibody.

It should be understood that the foregoing compositions and methods are readily adapted to methods described herein below for screening of and treatment with effective amounts of therapeutic agents directed to blocking T cell signaling (for example, through T cell receptors or its ligands), resulting in immunomodulation and/or enhancement of myelin repair.

An agent-induced change in gene expression or an agent-induced effect, may also be determined by detecting marker proteins. For example, marker proteins can be targets for immunostaining techniques known in the art to facilitate identification of cells (e.g., cell fate mapping). Non-limiting exemplary marker proteins of a myelinating cell (including oligodendrocyte and Schwann cell) may be selected from the group consisting of CC1, myelin basic protein (MBP), ceramide galactosyltransferase (CGT), myelin associated glycoprotein (MAG), myelin oligodendrocyte glycoprotein (MOG), oligodendrocyte-myelin glycoprotein (OMG), cyclic nucleotide phosphodiesterase (CNP), NOGO, myelin protein zero (MPZ), peripheral myelin protein 22 (PMP22), protein 2 (P2), galactocerebroside (GalC), sulfatide and proteolipid protein (PLP). MPZ, PMP22 and P2 are markers for Schwann cells.

If desired, cells (in culture or in vivo) can be modified to express fluorescent marker proteins, for example, so as to follow cell migration in vivo or in tissue culture. Non-exclusive examples of marker genes that can be used in the present invention include reef coral fluorescent proteins (RCFPs), HcRed1, AmCyan1, AsRed2, mRFP1, DsRed1, jellyfish fluorescent protein (FP) variants, red fluorescent protein, green fluorescent protein (GFP), blue fluorescent protein, luciferase, GFP mutant H9, GFP H9-40, EGFP, tetramethylrhodamine, Lissamine, Texas Red, EBFP, ECFP, EYFP, Citrine, Kaede, Azami Green, Midori Cyan, Kusabira Orange and naphthofluorescein, or enhanced functional variants thereof. Many genes encoding fluorophore proteins markers are known in the art, which markers are capable of use in the present invention. See, website: <cgr.harvard.edu/thornlab/gfps.htm>. Mutated version of fluorescence proteins that emit light of greater intensity or which exhibit wavelength shifts can also be utilized in the compositions and methods of the present invention; such variants are known in the art and commercially available. (See Clontech Catalogue, 2005).

Visualizing fluorescence (e.g., marker gene encoding a fluorescent protein) can be conducted with microscopy techniques, either through examining cell/tissue samples obtained from an animal (e.g., through sectioning and imaging using a confocal microscope), as well as examining living cells or detection of fluorescence in vivo. Visualization techniques include but are not limited utilization of confocal microscopy or photo-optical scanning techniques known in the art. Generally, fluorescence labels with emission wavelengths in the near-infrared are more amenable to deep-tissue imaging because both scattering and autofluorescence, which increase background noise, are reduced as wavelengths are increase. Examples of in vivo imaging are known in the art, such as disclosed by Mansfield et al., J. Biomed. Opt. 10:41207 (2005); Zhang et al., Drug Met. Disp. 31:1054-1064 (2003); Flusberg et al., Nat. Methods 2:941-950 2005); Mehta et al., Curr. Opin. Neurobiol. 14:617-628 (2004); Jung et al.; J. Neurophysiol. 92:3121-3133 (2004); U.S. Pat. Nos. 6,977,733 and 6,839,586, each disclosure of which is herein incorporated by reference.

B. Animal Models

In some aspects, screening assays for determining a beneficial therapeutically effective combination of agents directed to immunomodulation and myelin repair/remyelinaton or axonal protection are conducted utilizing animal models. In preferred embodiments, the animal is a small rodent, or simian species. In more preferred embodiments, the animal is a mouse, rat, guinea pig, or monkey.

In some embodiments, the animal is a transgenic animal that can be a “knock-out” or “knock-in”, with one or more desired characteristics. For example, in some embodiments, a transgenic animal can be modified to express or express at altered levels (i.e., up or down) an agent that promotes immunomodulation, myelin repair/remyelination or axonal protection. Therefore, such an animal is utilized to screen a plurality of different agents also directed to immunomodulation, myelin repair/remyelination or axonal protection, where if the transgenic animal comprises an agent directed to one end point, then the animal is administered an agent directed to a different end point(s), and vice versa, to identify a candidate combination of therapeutic agents that result in a synergistic therapeutic result for a neuropathy or related conditions described herein above.

As noted above, transgenic animals can be broadly categorized into two types: “knockouts” and “knockins”. A “knockout” has an alteration in the target gene via the introduction of transgenic sequences that results in a decrease of function of the target gene, preferably such that target gene expression is insignificant or undetectable. A “knockin” is a transgenic animal having an alteration in a host cell genome that results in an augmented expression of a target gene, e.g., by introduction of an additional copy of the target gene, or by operatively inserting a regulatory sequence that provides for enhanced expression of an endogenous copy of the target gene. The knock-in or knock-out transgenic animals can be heterozygous or homozygous with respect to the target genes. Both knockouts and knockins can be “bigenic”. Bigenic animals have at least two host cell genes being altered. A preferred bigenic animal carries a transgene encoding a neuronal cell-specific recombinase and another transgenic sequence that encodes neuronal cell-specific marker genes. The transgenic animals of the present invention can broadly be classified as Knockins.

In other embodiments, the transgenic model system can also be used for the development of a biologically active agents that promote or are beneficial for a neuronal remyelination. For example, a transgenic animal that is modified to express an agent resulting in an immunomodulatory, myelin repair or axonal protection phenotype, can be utilized in methods of screening unknown compounds to determine (1) if a compound enhances immune tolerance, suppresses an inflammatory response, or promotes remyelination and/or (2) if a compound can result in a synergistic therapeutic effect in the animal model. Moreover, neuronal cells can be isolated from the transgenic animals of the invention for further study or assays conducted in a cell-based or cell culture setting, including ex vivo techniques. Furthermore, the model system can be utilized to assay whether a test agent impart a detrimental effect or reduces remyelination, e.g., post demyelination insult.

For example, an animal may be administered an immunomodulatory agent such as anti-CD80(Fab) after a demyelinating condition. After demyelination, the animal is administered a candidate agent, such as a γ-secretase inhibitor, before, concurrent, or after administration of the immunomodulatory agent. The animal treated with anti-CD80(Fab) and the candidate agent is compared to animals administered the anti-CD80(Fab) alone and to animals administered the γ-secretase inhibitor alone. Candidate agents may be selected based on their synergistic affect with the immunomodulatory agent. Alternatively, an animal may be administered a myelin repair and/or axonal promoting agent after a demyelinating condition, and candidate agents that are immunomodulatory are administered before, concurrent with, or after administration of the myelin repair and/or axonal promoting agent. Candidate agents that provide a synergistic effects with the myelin repair and/or axonal promoting agent can be selected for further analysis.

Advances in technologies for embryo micromanipulation now permit introduction of heterologous DNA into fertilized mammalian ova as well. For instance, totipotent or pluripotent stem cells can be transformed by microinjection, calcium phosphate mediated precipitation, liposome fusion, retroviral infection or other means. The transformed cells are then introduced into the embryo, and the embryo will then develop into a transgenic animal. In a preferred embodiment, developing embryos are infected with a viral vector containing a desired transgene so that the transgenic animals expressing the transgene can be produced from the infected embryo. In another preferred embodiment, a desired transgene is coinjected into the pronucleus or cytoplasm of the embryo, preferably at the single cell stage, and the embryo is allowed to develop into a mature transgenic animal. These and other variant methods for generating transgenic animals are well established in the art and hence are not detailed herein. See, for example, U.S. Pat. Nos. 5,175,385 and 5,175,384.

Accordingly, in some embodiments the present invention provides a method of using animal models for detecting and quantifying synergistic combinatorial treatment. In one embodiment, the method comprises the steps of: (a) inducing demyelination insult in the transgenic animal of the invention expressing an immunotolerance-inducing agent; (b) administering a candidate agent and allowing time for myelin repair occur if it is to occur; (c) detecting and/or quantifying expression of cell-specific marker gene(s) (d) determining if and how much remyelination has occurred and if such remyelination is enhanced as compared to a control. In such an example, the control could be wild-type in which a disease model is induced, or a transgenic to which the candidate agent is not administered.

A number of methods for inducing demyelination in a test animal have been established. For instance, neuronal demyelination may be inflicted by pathogens or physical injuries, agents that induce inflammation and/or autoimmune responses in the test animal. The EAE model is a well studied animal model for human autoimmune diseases. Experimental allergic encephalomyelitis (EAE) is a mouse model for multiple sclerosis in which the rodent is immunized to specific myelin components. See, e.g., Popko et al., Mol. Neurobiol. 14:19-35 (1997); Popko and Baerwald, Neurochem. Res. 24:331-338 (1999); Steinman, Mult. Scler. 7:275-276 (2002). EAE can be induced in animals (usually mice but also rats, rabbits and monkeys) by injecting them with cells and tissues of the nervous system to trigger an immune response with some MS-like symptoms, such as weakness, paralysis, and incontinence. EAE is typically mediated by autoimmune CD4+ T-cells. These cells develop in the peripheral lymphoid organs and travel to the CNS causing an autoimmune response. The development of T cells is controlled largely by the expression of various cytokines as well as cellular adhesion molecules. The origin of the model is traced to the development of the rabies vaccine. Encephalomyopathy was caused in a small percentage of humans who received the rabies vaccine. Subsequent studies succeeded in inducing the paralytic disease in different animals including rabbits. Methods were developed to cause inflammatory reaction as well as demyelination with limited number of injections.

Furthermore, methods to induce a disease state can employ demyelination-inducing agents including but not limited to IFN-γ and cuprizone (bis-cyclohexanone oxaldihydrazone). The cuprizone-induced demyelination model is described in Matsushima et al., Brain Pathol. 11:107-116 (2002). In this method, the test animals are typically fed with a diet containing cuprizone for a few weeks ranging from about 1 to about 10 weeks.

After induction of a demyelination condition by an appropriate method, the animal may be allowed to recover for a sufficient amount of time to allow remyelination at or near the previously demyelinated lesions. While the amount of time required for developing remyelinated axons varies among different animals, it generally requires at least about 1 week, more often requires at least about 2 to 10 weeks, and even more often requires about 4 to about 10 weeks. Remeylination can be ascertained by observing an increase in myelinated axons in the nervous systems (e.g., in the central or peripheral nervous system), or by detecting an increase in the levels of marker proteins of a myelinating cell. The same methods of detecting demyelination can be employed to determine whether remyelination has occurred.

Animals may also be administered an agent prior, concurrent, or subsequent to demyelination. For example, an animal may be administered an immunomodulatory agent that suppresses the autoimmune response and compared to animals administered an immunomodulatory agent with a γ-secretase inhibitor, wherein the inhibitor is administered prior, concurrent, or subsequent to the immunomodulatory agent. Various amounts of the agents, different numbers of agents, and the time between administration of the agents and timing prior, concurrent, or subsequent to a demyelination are variables that may be performed to determine synergistic combinations of agents to promote remyelination.

### VI. THERAPEUTICS

A. Dosage

Depending on the patient and condition being treated and on the administration route, the peptides/polypeptides will generally be administered in dosages of 0.01 mg to 500 mg V/kg body weight per day, e.g. about 20 mg/day for an average person. The range is broad, since in general the efficacy of a therapeutic effect for different mammals varies widely with doses typically being 20, 30 or even 40 times smaller (per unit body weight) in man than in the rat. Similarly the mode of administration can have a large effect on dosage. Thus for example oral dosages in the rat may be ten times the injection dose. A typical dosage may be one injection daily. In some embodiments, dosage for one or a combination of agents can be from 0.01 to 5 mg, 1 to 10 mg, 5 to 20 mg, 10 to 50 mg, 20 to 100 mg, 50 to 150 mg, 100 to 250 mg, 150 to 300 mg, 250 to 500 mg, 300 to 600 mg or 500 to 100 mg V/kg body weight. In some embodiments, the dosage may be 20-2000 ug/dose, for example with anti-CD80(Fab).

Those of skill will readily appreciate that dose levels can vary as a function of the specific compound, the severity of the symptoms and the susceptibility of the subject to side effects. Some of the specific peptides are more potent than others. Preferred dosages for a given complex are readily determinable by those of skill in the art by a variety of means. A preferred means is to measure the physiological potency of a given compound.

In preferred embodiments, the immunomodulatory agents are co-administered at dosages determined to be therapeutic relative to the co-administered myelin-repair or axonal re-generation agent. In some embodiments, within one or more combinatorial method of the invention the immunoregulatory component comprises peptides or polypeptides, including but not limited to antibodies, APLs or Peptide-Coupled tolerance antigens, which are administered at dosages of 0.01 mg to 500 mg V/kg body weight per day. In preferred embodiments, patients receive 5 mg. In some embodiments, such agents are administered from between 3 to 5, 4 to 6, 5 to 7, or 6 to 10 consecutive days at the same or varying dosages. In some embodiments, the administration is repeated in a plurality of cycles, where each cycle comprises administration of an agent between 3 to 5, 4 to 7, 6 to 9, 7 to 10, 8 to 12, 9 to 16 or 10 to 21 days.

In some embodiments, antibodies for effecting immunomodulation are administered at dosages depending upon such factors as the patient's age, weight, height, sex, general medical condition and previous medical history. Typically, it is desirable to provide the recipient with a dosage of antibody component, immunoconjugate or fusion protein which is in the range of from about 1 pg/kg to 10 mg/kg (amount of agent/body weight of patient), although a lower or higher dosage also may be administered as circumstances dictate. Administration of antibodies (or any bioactive agents described herein) to a patient can be intravenous, intraarterial, intraperitoneal, intramuscular, subcutaneous, intrapleural, intrathecal, by perfusion through a regional catheter, or by direct intralesional injection. When administering therapeutic proteins by injection, the administration may be by continuous infusion or by single or multiple boluses. Intravenous injection provides a useful mode of administration due to the thoroughness of the circulation in rapidly distributing antibodies.

In other embodiments, the concentration of the therapeutically active antibody or antibody fragment (e.g., Fab or Fc portion) in a formulation may vary from about 0.1 to 100 weight %. In a preferred embodiment, the concentration of the antibody or antibody fragment is in the range of 0.003 to 1.0 molar. In order to treat a patient, a therapeutically effective dose of the antibody or antibody fragment may be administered. By “therapeutically effective dose” herein is meant a dose that produces the effects for which it is administered (e.g., blocking co-stimulation of T cells or B cells). The exact dose will depend on the purpose of the treatment, and will be ascertainable by one skilled in the art using known techniques. Dosages may range from 0.01 to 100 mg/kg of body weight or greater, for example 0.1, 1, 10, or 50 mg/kg of body weight, with 1 to 10 mg/kg being preferred. As is known in the art, adjustments for antibody or Fc fusion degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art.

Administration of the pharmaceutical composition comprising an antibody or antibody fragment, preferably in the form of a sterile aqueous solution, may be done in a variety of ways, including, but not limited to orally, subcutaneously, intravenously, intranasally, intraotically, transdermally, topically (e.g., gels, salves, lotions, creams, etc.), intraperitoneally, intramuscularly, intrapulmonary (e.g., AERx®. inhalable technology commercially available from Aradigm, or Inhance™ pulmonary delivery system commercially available from Inhale Therapeutics), vaginally, parenterally, rectally, or intraocularly. In some instances, for example for the treatment of wounds, inflammation, etc., the antibody or Fc fusion may be directly applied as a solution or spray. As is known in the art, the pharmaceutical composition may be formulated accordingly depending upon the manner of introduction.

In preferred embodiments, the antibodies are administered at low protein doses, such as 20 milligrams to 2 grams protein per dose, given once, or repeatedly, parenterally. Alternatively, antibodies are administered in doses of 20 to 1000 milligrams protein per dose, or 20 to 500 milligrams protein per dose, or 20 to 100 milligrams protein per dose. In some embodiments, such agents are administered from between 3 to 5, 4 to 7, 6 to 9, 7 to 10, 8 to 12, 9 to 16 or 10 to 21 days. In some embodiments, the administration is repeated in a plurality of cycles, where each cycle comprises administration of an agent between 3 to 5, 4 to 7, 6 to 9, 7 to 10, 8 to 12, 9 to 16 or 10 to 21 days.

The antibodies, alone or conjugated to liposomes, can be formulated according to known methods to prepare pharmaceutically useful compositions, whereby the therapeutic proteins are combined in a mixture with a pharmaceutically acceptable carrier. A composition is said to be a “pharmaceutically acceptable carrier” if its administration can be tolerated by a recipient patient. Sterile phosphate-buffered saline is one example of a pharmaceutically acceptable carrier. Other suitable carriers are well-known to those in the art. See, for example, REMINGTON'S PHARMACEUTICAL SCIENCES, 19th Ed. (1995).

For purposes of therapy, antibodies are administered to a patient in a therapeutically effective amount in a pharmaceutically acceptable carrier. In this regard, a “therapeutically effective amount” is one that is physiologically significant. An agent is physiologically significant if its presence results in a detectable change in the physiology of a recipient patient. In the present context, an agent is physiologically significant if its presence results in blocking immune cell activation, proliferation or differentiation. In preferred embodiments, the immune cells are T cells or B cells. Additional pharmaceutical methods may be employed to control the duration of action of an antibody in a therapeutic application. Control release preparations can be prepared through the use of polymers to complex or adsorb the antibody. For example, biocompatible polymers include matrices of poly(ethylene-co-vinyl acetate) and matrices of a polyanhydride copolymer of a stearic acid dimer and sebacic acid. Sherwood et al., Bio/Technology 10:1446 (1992). The rate of release of an antibody from such a matrix depends upon the molecular weight of the protein, the amount of antibody within the matrix, and the size of dispersed particles. Saltzman et al., Biophys. J. 55:163 (1989); Sherwood et al., supra. Other solid dosage forms are described in REMINGTON'S PHARMACEUTICAL SCIENCES, 19th ed. (1995).

B. Pharmaceutical Compositions

Pharmaceutical compositions are contemplated wherein a agent or agents is comprised of a peptide, polypeptide, aptamer, siRNA or antisense, antibody, antibody fragment, or small molecule of the present invention and one or more therapeutically active agents are formulated. Formulations of such agents are prepared for storage by mixing such agents having the desired degree of purity with optional pharmaceutically acceptable carriers, excipients or stabilizers (Remington's Pharmaceutical Sciences 16th edition, Osol, A. Ed., 1980), in the form of lyophilized formulations or aqueous solutions. Acceptable carriers, excipients, or stabilizers are nontoxic to recipients at the dosages and concentrations employed, and include buffers such as phosphate, citrate, acetate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl orbenzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; sweeteners and other flavoring agents; fillers such as microcrystalline cellulose, lactose, corn and other starches; binding agents; additives; coloring agents; salt-forming counter-ions such as sodium; metal complexes (e.g. Zn-protein complexes); and/or non-ionic surfactants such as TWEEN™, PLURONICS™ or polyethylene glycol (PEG).

In a preferred embodiment, the pharmaceutical composition that comprises the bioactive agents of the present invention is in a water-soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine. The formulations to be used for in vivo administration are preferrably sterile. This is readily accomplished by filtration through sterile filtration membranes or other methods known in the art.

The bioactive agents disclosed herein may also be formulated as immunoliposomes. A liposome is a small vesicle comprising various types of lipids, phospholipids and/or surfactant that is useful for delivery of a therapeutic agent to a mammal. Liposomes containing bioactive agents are prepared by methods known in the art, such as described in Epstein et al., Proc. Natl. Acad. Sci. USA 82:3688-3692 (1985); Hwang et al., Proc. Natl. Acad. Sci. USA 77:4030-4034 (1990); U.S. Pat. Nos. 4,485,045; 4,544,545; and PCT WO 97/38731. Liposomes with enhanced circulation time are disclosed in U.S. Pat. No. 5,013,556. The components of the liposome are commonly arranged in a bilayer formation, similar to the lipid arrangement of biological membranes. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. A chemotherapeutic agent or other therapeutically active agent is optionally contained within the liposome (Gabizon et al., J. National Cancer Inst 81:1484-1488 (1989).

The subject agents can also be formulated to yield a controlled-release formulation.

### EXAMPLES

### Example 1

### Screening Assay

The γ-secretase inhibitor LY411575 was administered in a cell-culture assay and illustrated that while there was no observed effect on T cell proliferation, the amount of T cell differentiation of the inflammatory Th1 subset of CD4⁺ T cells was decreased. When LY411575 was injected into EAE animals, the severity of EAE was decreased.

Candidate agents are screened with LY411575 in vitro, to identify agents that confers a synergistic effect of promoting myelination when compared to the candidate agent or LY411575 alone. Agents conferring synergistic effects are identified by increased oligodendrocyte proliferation, migration, or differentiation as compared to control cells.

Agents identified from in vitro assays are used in animal models. Relasping EAE (R-EAE), chronic EAE (C-EAE) or TMEV-IDD is induced in the appropriate mouse strains. Following onset of acute disease, the mice are separated equally by clinical disease scores into four groups: (1) mice receiving control agents; (2) mice receiving agent identified in in vitro screen; (3) mice receiving LY411575; or (4) combination of agent identified in in vitro screen and LY411575. Treatments are given as intraperitoneal injections from anywhere between 3 to 5, 4 to 6, 5 to 7, 6 to 8, 7 to 9, 8 to 10, 9 to 12, 10 to 14 or 12 to 16 days. The mice in various treatment groups are analyzed for both immune responses and CNS histology.

### Example 2

### Immunolopical Assays

Clinical disease scores are recorded daily to determine effects on clinical disease progression and relapse rate. CD4⁺ T cell responses are analyzed upon recall with the specific peptide used for priming. Delayed-type hypersensitivity (DTH) experiments are performed to determine antigen-specific CD4 Th1 activation and migration in vivo. In vitro recall experiments such as proliferation assays and ELISPOTS are performed to measure numbers of cytokine producing T cells. Cytokine LiquiChip analysis is performed to measure amount of cytokine production. Spleens and lymph nodes are isolated from treated and untreated mice to analyze immune responses upon re-challenge with myelin peptides.

Lower clinical scores may be expected in the combinatorial treatment group. Amelioration of clinical disease may result in a lower Th1 cytokine expression (i.e., IFN-γ, TNF-α, IL-2) and higher Th2 expression (i.e., IL-4, IL-5, IL-10, TGF-β). Flow cytometry (FACS) and immunohistochemistry is also performed to analyze the numbers of CD4⁺ T cells, macrophages and dendritic cells infiltrating into the CNS; and Agilent gene chip array analysis of CNS tissue comparing the various treatment groups. Preliminary data indicates lower numbers and expression of T cells in the CNS by FACS and immunohistochemistry, and decreased infiltrating dendritic cells (DC) and macrophages (Mφ) in the combined treatment group.

### Example 3

### Neurobiological Experiments

PLP staining is combined with staining for CD4⁺ T cells and CD11b⁺ macrophages to identify myelin and extent of infiltration following the various treatments. Additionally, CNPase and CC1, markers of oligodendrocyte lineage cells, are used in immunohistochemical analyses to detect differences in oligodendrocyte numbers between treated and control mice. In addition to oligodendrocyte differentiation, which is only one component of successful myelin repair, toluidine blue and/or luxol fast blue staining procedures are used to detect the extent of remyelination in fixed sections of brain and spinal cord. Where combinatorial treatment enhances remyelination (as assessed by toluidine or luxol fast blue), correlation with increased myelin gene expression is determined by real-time PCR and microarray analysis).

### Example 4

### Combinatorial Therapy with anti-CD80 and DAPT

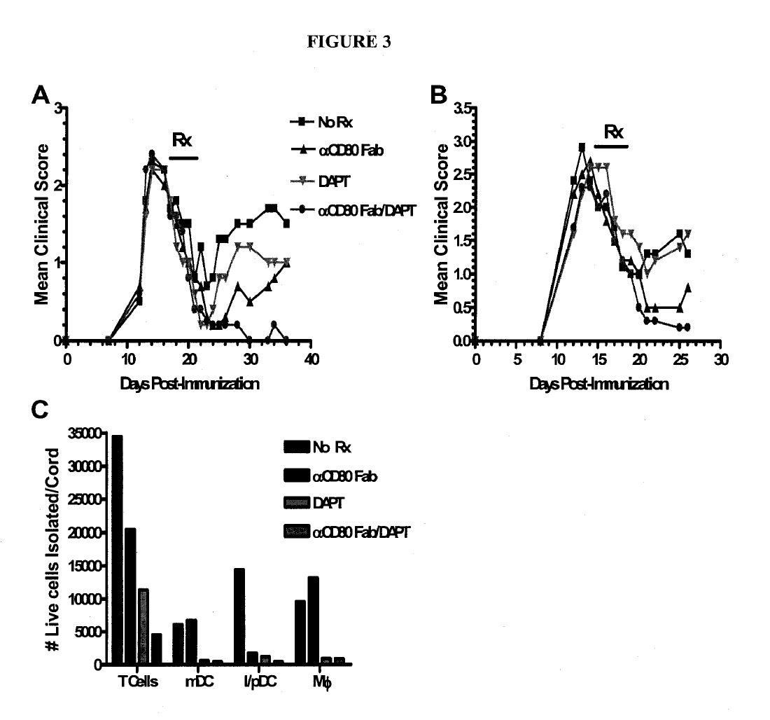

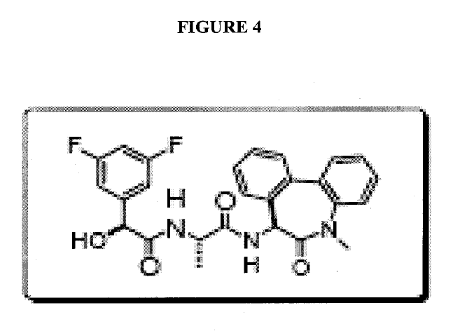

Demyelinating insult was induced in a mouse model by immunization with PLP₁₃₉₋₁₅₁. At the peak of the acute phase of the disease (day 15-16 post-immunization), the mice were separated into four treatment groups which received: (1) five daily intraperitoneal treatments with 50 μg of control antibody; (2) five daily i.p. treatments with 50 μg of anti-CD80 Fab; (3) five daily i.p. treatments with DAPT 100 μg; or (4) five daily i.p. treatments with both anti-CD80Fab and DAPT. The results indicate that there is a significant synergistic therapeutic effect that is both protective and enhances a recovery effect on progression of clinical paralysis in mice treated with both anti-CD80Fab and DAPT in combination compared to treatment with either anti-CD80 Fab or DAPT alone (FIG. 3B). Flow cytometric analysis of the number of CNS infiltrating cells from the treated mice show that the combined therapy resulted in substantially reduced numbers of T cells, myeloid dendritic cells (mDC), lymphoid/plasmacytoid dendritic cells (1/p DC), and macrophages (Mφ) in the combined treatment group (FIG. 3C).

Co-administration of anti-CD80(Fab) and DAPT represent one embodiment of the various bioactive agents that can be utilized in the combinatorial methods described herein. Furthermore, the R-EAE model is one of many suitable models that can be utilized, including C-EAE and TMEV-IDD.

### Example 5

### Combinatorial Therapy with Peptide-Coupled Cell Tolerance and LY411,575

R-EAE mice are intravenously injected with splenocytes coupled to priming peptide (to block onset of disease), the spread epitopes (to block specific relapses) or a combination of myelin peptides. Splenocytes are coupled to the peptides by using the ethylene carbodiimide (ECDI) procedure. The mice are also administered LY411,575 either before, during, or after injection of the myelin peptide-pulsed, ECDI-fixed splenocytes and compared to the mice not administered LY411,575 and mice administered LY411,575 but not injected with myelin peptide pulsed, ECDI-fixed splenocytes to determine extent of remyelination and ongoing EAE symptoms.

### Example 6

### Combinatorial Therapy with anti-CD80 and rHIgM22

Demyelinating insult is induced in a mouse model by immunization with PLP₁₃₉₋₁₅₁. At the peak of the acute phase of the disease (day 15-16 post-immunization), the mice are separated into four treatment groups which received: (1) five daily intraperitoneal treatments with of control antibody; (2) five daily i.p. treatments with anti-CD80Fab; (3) five daily i.p. treatments with rHIgM22; or (4) five daily i.p. treatments with both anti-CD80Fab and rHIgM22. The protective and recovery effect on progression of clinical paralysis in mice treated with both anti-CD80Fab and rHIgM22 in combination compared to treatment with either anti-CD80Fab or rHIgM22 alone is determined. Flow cytometric analysis of the number of CNS infiltrating cells from the treated mice is performed determine the number of T cells, myeloid dendritic cells (mDC), lymphoid/plasmacytoid dendritic cells (1/p DC), and macrophages (Mφ) in the different groups. Co-administration of anti-CD80(Fab) and rHIgM22 is likely to have a synergistic therapeutic effect in promoting protective and recovery effects on progression of clinical paralysis with decreased numbers of T cells, myeloid dendritic cells (mDC), lymphoid/plasmacytoid dendritic cells (1/p DC), and macrophages (Mφ).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the claims herein define the scope of the invention and that methods and structures within the scope of these claims and their equivalents be covered thereby.

What is claimed is:

1. A composition for treating a demyelinating condition comprising:
a) a therapeutically effective amount of a first agent, wherein said first agent is immunomodulatory; and,
b) a therapeutically effective amount of a second agent, wherein said second agent promotes myelin repair,
wherein administering said first and second agents result in a synergistic therapeutic effect for treating said demyelinating condition.

2. The composition of claim 1, wherein said first and second agents are present in synergistic amounts.

3. A composition for treating a demyelinating condition comprising:
a) a therapeutically effective amount of a first agent, wherein said first agent is immunomodulatory;
b) a therapeutically effective amount of a second agent, wherein said second agent promotes oligodendrocyte differentiation, and,
c) a therapeutically effective amount of a third agent, wherein said third agent promotes oligodendrocyte proliferation;
wherein administering said first, second and third agents result in a synergistic therapeutic effect for treating said demyelinating condition.

4. The composition of claim 1 or 3, wherein said synergistic effect is more than 1 fold than the therapeutic effect of said first agent alone or said second agent alone.

5. The composition of claim 1 or 3, wherein said demyelinating condition is multiple sclerosis.

6. The composition of claim 1 or 3, wherein said first agent suppresses the autoimmune response.

7. The composition of claim 1 or 3, wherein said first agent targets T-cells, plasma cells, or macrophages.

8. The composition of claim 1 or 3, wherein said first agent inhibits T-cell receptor signaling in an autoimmune response.

9. The composition of claim 1 or 3, wherein said first agent or said second agent is selected from the group consisting of: an altered peptide ligand, peptide-coupled cell, antisense molecule, siRNA, aptamer, small molecule and antibody.

10. The composition of claim 1 or 3, wherein said first agent is specific for a ligand, or its receptor, wherein said ligand is selected from the group consisting of: CD80, CD86, CD28, CD40L, CD3, CD4, CD22, CD25, CD40, CD44, CD45, CD45RB, CD49, CD62, CD69, and CD154.

11. The composition of claim 1 or 3, wherein said first agent is a CD80 antibody or CD3 antibody.

12. The composition of claim 1 or 3, wherein said second agent inhibits Notch signaling.

13. The composition of claim 1 or 3, wherein said second agent is an IgM antibody.

14. The composition of claim 1 or 3, wherein said second agent is a γ-secretase inhibitor.

15. The composition of claim 1 or 3, wherein said second agent is selected from a group consisting of: DAPT, Ly411575, III-31-C, and rHIgM22.

16. A method for treating a demyelinating condition comprising administering to a subject in need thereof:
a) a therapeutically effective amount of a first agent, wherein said first agent is immunomodulatory; and,
b) a therapeutically effective amount of a second agent, wherein said second agent promotes remyelination,
wherein administering said first and second agents result in a synergistic therapeutic effect in promoting remyelination.

17. A method of promoting remyelination comprising:
a) contacting a cell in a co-culture with a first agent, wherein said first agent is immunomodulatory,
b) contacting said cell with a second agent, wherein said second agent promotes remyelination,
wherein contacting said cell with said first and second agents result in a synergistic effect in promoting remyelination.

18. A method for treating a demyelinating condition comprising administering to a subject in need thereof:
a) a therapeutically effective amount of a first agent, wherein said first agent is immunomodulatory;
b) a therapeutically effective amount of a second agent, wherein said second agent promotes oligodendrocyte differentiation, and,
c) a therapeutically effective amount of a third agent, wherein said third agent promotes oligodendrocyte proliferation,
wherein administering said first, second and third agents result in a synergistic therapeutic effect for treating said demyelinating condition.

19. The method of claim 16 or 17, wherein said first agent and said second agent are not administered concurrently.

20. The method of claim 16 or 17, wherein said first agent is administered concurrent with said second agent

21. The method of claim 16, 17, or 18, wherein said synergistic effect is more than 1 fold greater than the therapeutic effect of said first agent alone or said second agent alone.

22. The method of claim 16 or 17, wherein said first agent or said second agent is selected from the group consisting of: an altered peptide ligand, peptide-coupled cell, antisense molecule, siRNA, aptamer, small molecule and antibody.

23. The method of claim 16 or 17, wherein said first agent is specific for a ligand, or its receptor, wherein said ligand is selected from the group consisting of: CD80, CD86, CD28, CD40L, CD3, CD4, CD22, CD25, CD40, CD44, CD45, CD45RB, CD49, CD62, CD69, and CD154.

24. The method of claim 16, 17, or 18, wherein said first agent is a CD80 antibody or a CD3 antibody.

25. The method of claim 16, 17, or 18, wherein said second agent inhibits Notch signaling.

26. The method of claim 16, 17, or 18, wherein said second agent is an IgM antibody or γ-secretase inhibitor.

27. The method of claim 16, 17, or 18, wherein said second agent is selected from a group consisting of DAPT, Ly411575, III-31-C, and rHIgM22.

28. The method of claim 16, 17, or 18, wherein said demyelinating condition is multiple sclerosis.

29. The method of claim 16, 17, or 18, wherein said first agent suppresses an autoimmune response.

30. The method of claim 16 or 17, wherein said first agent targets T-cells, plasma cells, or macrophages.

31. The method of claim 16, 17, or 18, wherein said first agent inhibits T-cell receptor signaling in an autoimmune response.

32. The method of claim 17, wherein said contacting occurs in vitro.

33. The method of claim 17, wherein said contacting occurs in vivo.
